(19) United States
(12) Patent Application Publication
(10) Pub. No.: US 2012/0046364 A1
(43) Pub. Date: Feb. 23, 2012

(54) Novel Sulfonic Acid-Containing Thyromimetics, and Methods for Their Use

(75) Inventors: Serge H. Boyer, San Diego, CA (US); Scott J. Hecker, Del Mar, CA (US); Mali Venkat Reddy, Cupertino, CA (US)

(73) Assignee: METABASIS THERAPEUTICS, INC., La Jolla, CA (US)

(21) Appl. No.: 13/148,709

(22) PCT Filed: Feb. 9, 2010
(86) PCT No.: PCT/US10/23560

§ 371 Date: Nov. 7, 2011

Related U.S. Application Data
(60) Provisional application No. 61/151,312, filed on Feb. 10, 2009.

(51) Int. Cl. A61K 31/185; C07C 309/42; A61P 3/00; A61P 3/04; A61P 7/00; A61P 3/10; A61P 9/10; A61P 9/00; A61P 9/12; A61P 1/16; A61P 3/08; C07C 309/24; A61P 3/06

(52) U.S. Cl. 514/576; 562/42; 562/74

(57) ABSTRACT

The present invention relates to sulfonic acid containing compounds of formula IB, in which G, R¹, R³ and T are as defined in the claims, that bind to thyroid receptors in the liver. Activation of these receptors results in modulation of gene expression of genes regulated by thyroid hormones. The compounds can be used to treat diseases and disorders including metabolic diseases such as obesity, NASH, hypercholesterolemia and hyperlipidemia, as well as associated conditions such as atherosclerosis, coronary heart disease, impaired glucose tolerance, metabolic syndrome X and diabetes.

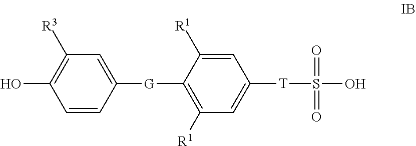

### RELATED APPLICATIONS

This application claims the benefit of the filing date of U.S. Provisional Application No. 61/151,312, filed on Feb. 10, 2009, the contents of which are herein incorporated by reference in their entirety.

### FIELD OF THE INVENTION

The present invention is directed toward novel sulfonic acid-containing compounds and methods of their use.

### BACKGROUND OF THE INVENTION

Thyroid hormones (TH) are synthesized in the thyroid in response to thyroid stimulating hormone (TSH), which is secreted by the pituitary gland in response to various stimulants (e.g., thyrotropin-releasing factor (TRF) from the hypothalamus). Thyroid hormones are iodinated O-aryl tyrosine analogues excreted into the circulation primarily as T4. T4 is rapidly deiodinated in the liver and kidney by thyroxine 5′-deiodinase to T3, which is the most potent TH. T3 is metabolized to inactive metabolites via a variety of pathways, including pathways involving deiodination, glucuronidation, sulfation, deamination, and decarboxylation. Most of the metabolic pathways reside in the liver.

THs have profound physiological effects in animals and humans. Hyperthyroidism is associated with increased body temperature, general nervousness, weight loss despite increased appetite, muscle weakness and fatigue, increased bone resorption and enhanced calcification, and a variety of cardiovascular changes, including increased heart rate, increased stroke volume, increased cardiac index, cardiac hypertrophy, decreased peripheral vascular resistance, and increased pulse pressure. Hypothyroidism is generally associated with the opposite effects.

The biological activity of THs is mediated largely through thyroid hormone receptors (TRs). TRs belong to the receptor superfamily known as nuclear receptors, which, along with its common partner, the retinoid X receptor, form heterodimers that act as ligand-inducible transcription factors. Like other nuclear receptors, TRs have a ligand binding domain and a DNA binding domain and regulate gene expression through ligand-dependent interactions with DNA response elements (thyroid response elements, TREs). Currently, the literature shows that TRs are encoded by two distinct genes (TRα and TRβ), which produce several isoforms through alternative splicing (Williams, Mol Cell Biol. 20(22):8329-42 (2000); Nagaya, et al., Biochem Biophys Res Commun 226(2):426-30 (1996)). The major isoforms that have so far been identified are TRα-1, TRα-2, TRβ-1 and TRβ-2. TRα-1 is ubiquitously expressed in the rat with highest expression in skeletal muscle and brown fat. TRβ-1 is also ubiquitously expressed with highest expression in the liver, brain and kidney. TRβ-2 is expressed in the anterior pituitary gland and specific regions of the hypothalamus as well as the developing brain and inner ear. In the rat and mouse liver, TRβ-1 is the predominant isoform (80%). The TR isoforms found in human and rat are highly homologous with respect to their amino acid sequences which suggest that each serves a specialized function.

TSH is an anterior pituitary hormone that regulates thyroid hormone production. TSH formation and secretion is in turn regulated by the hypothalamic thyrotropin releasing factor (TRF). TSH controls the uptake of iodide by the thyroid, the subsequent release of iodinated thyronines from thyroglobulin (e.g., T3, T4) as well as possibly the intrapituitary conversion of circulating T4 to T3. Compounds that mimic T3 and T4 can negatively regulate both TSH and TRF secretion resulting in suppression of TSH levels and decreased levels of T3 and other iodinated thyronines. Negative regulation of TSH is postulated based on co-transfection and knockout studies (Abel et al., J. Clin. Invest., 104, 291-300, (1999)) to arise through activation of the thyroid receptor TRβ, possibly the isoform TRβ-2, which is highly expressed in the pituitary.

The most widely recognized effects of THs are an increase in metabolic rate, oxygen consumption and heat production. T3 treatment increases oxygen consumption in isolated perfused liver and isolated hepatocytes. (Oh, et al., J. Nutr. 125(1):112-24 (1995); Oh, et al., Proc. Soc. Exp. Biol. Med. 207(3): 260-7 (1994)) Liver mitochondria from hyperthyroid rats exhibit increased oxygen consumption (Carreras, et al., Am J Physiol Heart Circ Physiol. 281(6):H2282-8 (2001) and higher activities of enzymes in the oxidative pathways (Dummler et al., Biochem J. 317(3):913-8 (1996), Schmehl, et al., FEBS Lett. 375(3):206-10 (1995), Harper et al., Can J Physiol Pharmacol. 72(8):899-908 (1994)). Conversely, mitochondria from hypothyroid rats show decreased oxygen consumption. Increased metabolic rates are associated with increased mitochondrialgenesis and the associated 2 to 8-fold increase in mitochondrial mRNA levels. Some of the energy produced from the increased metabolic rate is captured as ATP (adenosine 5′-triphosphate), which is stored or used to drive biosynthetic pathways (e.g., gluconeogenesis, lipogenesis, lipoprotein synthesis). Much of the energy, however, is lost in the form of heat (thermogenesis), which is associated with an increase in mitochondrial proton leak possibly arising from TH-mediated effects on mitochondrial membrane, uncoupling proteins, enzymes involved in the inefficient sn-glycerol 3-phosphate shuttle such as mitochondrial sn-glycerol 3-phosphate dehydrogenase (mGPDH), and/or enzymes associated with proton leakage such as the adenine nucleotide transporter (ANT), Na⁺/K⁺-ATPase, Ca²⁺-ATPase and ATP synthase.

THs also stimulate metabolism of cholesterol to bile acids. Hyperthyroidism leads to decreased plasma cholesterol levels, which is likely due to increased hepatic LDL receptor expression. Hypothyroidism is a well-established cause of hypercholesterolemia and elevated serum LDL. L-T3 is known to lower plasma cholesterol levels. The effects of T3 are attributed to TRβ since TRβ-deficient mice are resistant to T3-induced reduction in cholesterol levels. The effects on cholesterol levels have been postulated to result from direct effects on LDL receptor expression, enzymes involved in conversion of cholesterol to bile acids such as the rate-limiting enzyme cholesterol 7α-hydroxylase (CYP7A) and/or possibly enzymes involved in cholesterol synthesis such as HMG CoA reductase. In addition, THs are known to affect levels of other lipoproteins linked to atherosclerosis. THs stimulate apo AI and the secretion of apo AI in HDL while reducing apo AII. Accordingly, one would expect T3 and T3 mimetics to inhibit the atherosclerotic process in the cholesterol fed animal.

THs simultaneously increase de novo fatty acid synthesis and oxidation through effects on enzymes such as ACC, FAS, and spot-14. THs increase circulating free fatty acids (FFA) levels in part by increasing production of FFAs from adipose tissue via TH-induced lipolysis. In addition, THS increase mitochondrial enzyme levels involved in FFA oxidation, e.g., carnitine palmitoyltransferase 1 (CPT-1) and enzymes involved in energy storage and consumption.

The liver represents a major target organ of THs. Microarray analysis of hepatic gene expression from livers of hypothyroid mice and mice treated with T3 showed changes in mRNA levels for 55 genes (14 positively regulated and 41 negatively regulated) (Feng, et al., Mol. Endocrinol. 14(7): 947-55 (2000). Others have estimated that approximately 8% of the hepatic genes are regulated by T3. Many of these genes are important to both fatty acid and cholesterol synthesis and metabolism. T3 is also known to have other effects in liver, including effects on carbohydrates through increased glycogenolysis and gluconeogenesis and decreased insulin action.

The heart is also a major target organ of THs. THs lower systemic vascular resistance, increase blood volume and produce inotropic and chronotropic effects. Overall TH results in increased cardiac output, which may suggest that T3 or T3 mimetics might be of use to treat patients with compromised cardiac function (e.g., patients undergoing coronary artery bypass grafting (CABG) or cardiac arrest) (U.S. Pat. No. 5,158,978). The changes in cardiac function are a result of changes in cardiac gene expression. Increased protein synthesis and increased cardiac organ weight are readily observed in T3-treated animals and represent the side effect of T3 that limits therapeutic use. TRβ knockout mice exhibit high TSH and T4 levels and increased heart rate suggesting that they retain cardiac sensitivity and therefore that the cardiac effects are via TRα. TRα knockouts exhibit reduced heart rates.

THs also play a role in the development and function of brown and white adipose tissue. Both TRα and TRβ are expressed in brown adipose tissue (BAT). THs induce differentiation of white adipose tissue (WAT) as well as a variety of lipogenic genes, including ACC, FAS, glucose-6-phosphate dehydrogenase and spot-14. Overall THs play an important role in regulating basal oxygen consumption, fat stores, lipogenesis and lipolysis (Oppenheimer, et al., J. Clin. Invest. 87(1): 125-32 (1991)).

TH has been used as an antiobesity drug for over 50 years. In the 1940s TH was used alone, whereas in the 1950s it was used in combination with diuretics and in the 1960s in combination with amphetamines. Hyperthyroidism is associated with increased food intake but is also associated with an overall increase in the basal metabolic rate (BMR). Hyperthyroidism is also associated with decreased body weight (ca. 15%) whereas hypothyroidism is associated with a 25-30% increase in body weight. Treating hypothyroidism patients with T3 leads to a decrease in body weight for most patients but not all (17% of the patients maintain weight).

The effectiveness of TH treatment is complicated by the need for supraphysiological doses of T3 and the associated side effects, which include cardiac problems, muscle weakness and excess erosion of body mass. Long-term therapy has also been associated with bone loss. With these side effects, the medical community has tended to use thyroxine at low doses as an adjunct to dietary treatments. At these doses, TH has little effect on body weight or BMR.

The effectiveness of T3 to induce weight loss may be attenuated by defects in TH action. In comparison to normal animals, higher T3 doses were required in ob/ob mice to affect oxygen consumption, which was only observed in muscle, with no changes in liver and BAT. (Oh, et al., J. Nutr. 125(1): 112-24 (1995); Oh, et al., Proc. Soc. Exp. Biol. Med. 207(3): 260-7 (1994)). These effects were at least partially attributed to decreased uptake of T3 by the liver.

T3 analogues have been reported. Many were designed for use as cholesterol-lowering agents. Analogues that lower cholesterol and various lipoproteins (e.g., LDL cholesterol and Lp(a)) without generating adverse cardiac effects have been reported (e.g., Underwood, et al., Nature 324: 425-9 (1986)). In some cases the improved therapeutic profile is attributed to increased specificity for the TR-β wherein other cases it may be due to enhanced liver distribution. (Stanton, et al., Bioorg. Med. Chem. Lett. 10(15): 1661-3 (2000); Dow et al., Bioorg. Med. Chem. Lett., 13(3): 379-82 (2003)).

T3 and T3 mimetics are thought to inhibit atherosclerosis by modulating the levels of certain lipoproteins known to be independent risk factors or potential risk factors of atherosclerosis, including low density lipoprotein (LDL)-cholesterol, high density lipoprotein (HDL)-cholesterol, apoAI, which is a major apoprotein constituent of high density lipoprotein (HDL) particles and lipoprotein (a) or Lp (a).

Lp(a) is an important risk factor, elevated in many patients with premature atherosclerosis. Lp(a) is considered highly atherogenic (de Bruin et al., J. Clin. Endo. Metab., 76, 121-126 (1993)). In man, Lp(a) is a hepatic acute phase protein that promotes the binding of LDL to cell surfaces independent of LDL receptors. Accordingly, Lp(a) is thought to provide supplementary cholesterol to certain cells, e.g., cells involved in inflammation or repair. Lp(a) is an independent risk factor for premature atherosclerosis. Lp(a) is synthesized in the liver.

Apolipoprotein AI or apoAI is the major component of HDL, which is an independent risk factor of atherosclerosis. apoAI is thought to promote the efflux of cholesterol from peripheral tissues and higher levels of HDL (or apoAI) result in decreased risk of atherosclerosis.

Hyperthyroidism worsens glycemic control in type 2 diabetics. TH therapy is reported to stimulate hepatic gluconeogenesis. Enzymes specific to gluconeogenesis and important for controlling the pathway and its physiological role of producing glucose are known to be influenced by TH therapy. Phosphoenolpyruvate carboxykinase (PEPCK) is upregulated by TH (Park et al, J. Biol. Chem., 274, 211 (1999)) whereas others have found that glucose 6-phosphatase is upregulated (Feng et al., Mol. Endrocrinol., 14, 947 (2000)). TH therapy is also associated with reduced glycogen levels.

TH therapy results in improved non insulin stimulated and insulin stimulated glucose utilization and decreased insulin resistance in the muscle of ob/ob mice. (Oh et al., J. Nutr., 125, 125 (1995)).

There is still a need for novel thyromimetics that can be used to modulate cholesterol levels, to treat obesity, and other metabolic disorders especially with reduced undesirable effects.

Thus, there remains a need to develop characterize and optimize lead molecules for the development of novel drugs for treating or preventing diseases associated with nonsense mutations of mRNA. Accordingly, it is an object of the present invention to provide such compounds.

All documents referred to herein are incorporated by reference into the present application as though fully set forth herein.

### SUMMARY OF THE INVENTION

In accordance with the present invention, novel sulfonic-acid compounds have been identified, and methods for their use provided.

In certain aspects, sulfonic acid-containing compounds that are thyroid receptor ligands, pharmaceutically acceptable salts, and to prodrugs of these compounds as well as their preparation and uses for preventing and/or treating metabolic diseases such as obesity, NASH, hypercholesterolemia and hyperlipidemia as well as associated conditions such as atherosclerosis, coronary heart disease, impaired glucose tolerance and diabetes.

In other aspects, the invention also relates to the liver specific delivery of thyroid receptor ligands and the use of these compounds for the prevention and treatment of diseases responsive to modulation of T3-responsive genes in the liver.

In another aspect of the invention, compounds of Formula I, IA and IB, as described herein, are provided, as well as methods of their use.

These and other aspects of the invention will be more clearly understood with reference to the following preferred embodiments and detailed description.

### DETAILED DESCRIPTION OF THE INVENTION

Thyroid hormones and thyroid hormone mimetics bind to thyroid hormone receptors in the nucleus of cells and can change expression levels of genes encoding proteins that play an important role in metabolic diseases. Metabolic diseases that can be prevented or treated with thyroid hormone mimetics include obesity and lipid disorders such as hypercholesterolemia, hyperlipidemia, and hypertriglyceridemia as described in further detail below. Other metabolic diseases that can be prevented or treated with thyroid hormone mimetics include fatty liver/steatosis, NAFLD, NASH, diabetes, impaired glucose tolerance, and insulin resistance. Conditions associated with these diseases, such as atherosclerosis, coronary artery disease, and heart failure, can also be treated with these thyroid hormone receptor binding compounds.

Prior to the discoveries of the present invention, sulfonic acids were thought to be a poor replacement for carboxylic acids based on differences in geometry, size, and charge. Sulfonic acids can also display differences in cellular and in vivo potency, oral bioavailability, pharmacokinetics, metabolism, and safety. T3 and previously reported T3 mimetics contain a carboxylic acid thought to be important for binding and activation of T3 responsive genes. The carboxylic acid may also be important in the transport and distribution of these compounds through various transport proteins. Transport proteins can enhance transport of certain compounds, particularly negatively charged compounds, to the nucleus.

Nonetheless, it was unexpectedly found in accordance with the present invention that the sulfonic acid containing compounds described herein have demonstrated oral efficacy. Such finding was unexpected as sulfonic acids are highly charged species at physiological pH and are not expected to be orally active.

In certain aspects, the present invention relates to methods of preventing or treating metabolic diseases with sulfonic acid-containing compounds, pharmaceutically acceptable salts and prodrugs thereof, and pharmaceutically acceptable salts of the prodrugs, where the sulfonic acid-containing compounds bind to a thyroid hormone receptor.

In other aspects, the present invention relates to sulfonic acid containing compounds that bind to thyroid receptors in the liver. Activation of these receptors results in modulation of gene expression of genes regulated by thyroid hormones.

The present invention also relates to pharmaceutically acceptable salts and co-crystals, prodrugs, and pharmaceutically acceptable salts and co-crystals of these prodrugs of these compounds.

The compounds can be used to treat diseases and disorders including metabolic diseases. In one aspect, the sulfonic acid-containing compounds are useful for improving efficacy, improving the therapeutic index, e.g., decreasing non-liver related toxicities and side effects, or for improving liver selectivity, i.e., increasing distribution of an active drug to the liver relative to extrahepatic tissues and more specifically increasing distribution of the an active drug to the nucleus of liver cells relative to the nucleus of extrahepatic tissue cells (including heart, kidney and pituitary).

Prodrugs of the sulfonic acid-containing compounds are useful for increasing oral bioavailability and sustained delivery of the sulfonic acid-containing compounds.

Definitions

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g., the phenol group of the compounds described herein, that cleave in vivo. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, and/or pharmacodynamic half-life, etc.

Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

As used herein, the term “alkyl” generally refers to saturated hydrocarbyl radicals of straight, branched or cyclic configuration including methyl, ethyl, n-propyl, isopropyl, n-butyl, isobutyl, sec-butyl, tert-butyl, n-pentyl, n-hexyl, cyclohexyl, n-heptyl, octyl, n-octyl, and the like. In some embodiments, alkyl substituents may be C₁ to C₂₀, C₁ to C₁₂, C₁ to C₈, C₁ to C₆, or C₁ to C₄ alkyl groups. In certain embodiments, the alkyl group may be optionally substituted. For instance, the alkyl group may be a haloalkyl, including monohaloalkyl, dihaloalkyl, and trihaloalkyl.

As used herein, “alkylene” generally refers to linear, branched or cyclic alkene radicals having one or more carbon-carbon double bonds, such as C₂ to C₆ alkylene groups including 3-propenyl. Again, in certain embodiments, the alkyl group may be optionally substituted.

The term “alkenyl” refers to unsaturated groups which have, e.g., 2 to 12 atoms and contain at least one carbon-carbon double bond and includes straight-chain, branched-chain and cyclic groups. Alkenyl groups may be optionally substituted. Suitable alkenyl groups include allyl.

The term “alkynyl” refers to unsaturated groups which have, e.g., 2 to 12 atoms and contain at least one carbon-carbon triple bond and includes straight-chain, branched-chain and cyclic groups. Alkynyl groups may be optionally substituted. Suitable alkynyl groups include ethynyl.

As used herein, “aryl” refers to a carbocyclic aromatic ring structure. Included in the scope of aryl groups are aromatic rings having from five to twenty ring atoms. Aryl ring structures include compounds having one or more ring structures, such as mono-, bi-, or tricyclic compounds, and includes carbocyclic aryl and heterocyclic aryl and biaryl groups. Examples of aryl groups that include phenyl, tolyl, anthracenyl, fluorenyl, indenyl, azulenyl, phenanthrenyl (i.e., phenanthrene), and napthyl (i.e., napthalene) ring structures. Again, in certain embodiments, the alkyl group may be optionally substituted.

As used herein, “heterocycle” refers to cyclic ring structures in which one or more atoms in the ring, the heteroatom(s), is an element other than carbon. Heteroatoms are typically O, S or N atoms. Included within the scope of heterocycle, and independently selectable, are O, N, and S heterocycle ring structures. The ring structure may include compounds having one or more ring structures, such as mono-, bi-, or tricyclic compounds, and may be aromatic, i.e., the ring structure may be a heteroaryl. Example of heterocyclo groups include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl or tetrahydrothiopyranyl and the like. Again, in certain embodiments, the alkyl group may be optionally substituted.

As used herein, “heteroaryl” refers to cyclic aromatic ring structures in which one or more atoms in the ring, the heteroatom(s), is an element other than carbon. Heteroatoms are typically O, S or N atoms. Included within the scope of heteroaryl, and independently selectable, are O, N, and S heteroaryl ring structures. The ring structure may include compounds having one or more ring structures, such as mono-, bi-, or tricyclic compounds. In some embodiments, the heteroaryl groups may be selected from heteroaryl groups that contain two or more heteroatoms, three or more heteroatoms, or four or more heteroatoms. Heteroaryl ring structures may be selected from those that contain five or more atoms, six or more atoms, or eight or more atoms. In a preferred embodiment, the heteroaryl including five to ten atoms. Examples of heteroaryl ring structures include: acridine, benzimidazole, benzoxazole, benzodioxole, benzofuran, 1,3-diazine, 1,2-diazine, 1,2-diazole, 1,4-diazanaphthalene, furan, furazan, imidazole, indole, isoxazole, isoquinoline, isothiazole, oxazole, purine, pyridazine, pyrazole, pyridine, pyrazine, pyrimidine, pyrrole, quinoline, quinoxaline, thiazole, thiophene, 1,3,5-triazine, 1,2,4-triazine, 1,2,3-triazine, tetrazole and quinazoline. Again, in certain embodiments, the alkyl group may be optionally substituted.

As used herein, “alkoxy” generally refers to a group with the structure —O—R. In certain embodiments, R may be an alkyl group, such as a C₁ to C₈, C₁ to C₆ alkyl group, or C₁ to C₄ alkyl group. In certain embodiments, the R group of the alkoxy may optionally be substituted, e.g., with at least one halogen. For example, the R group of the alkoxy may be a haloalkyl, i.e., haloalkoxy.

Halogen substituents may be independently selected from the halogens such as fluorine, chlorine, bromine, iodine, and astatine.

More specifically, the term “optionally substituted” or “substituted” includes groups substituted by one, two, three, four, five, or six substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, -carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower-carboxamidoalkylaryl, lower-carboxamidoaryl, lower hydroxyalkyl, lower haloalkyl, lower alkylaminoalkylcarboxy-, lower aminocarboxamidoalkyl-, cyano, lower alkoxyalkyl, lower perhaloalkyl, and lower arylalkyloxyalkyl.

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

The term “pharmaceutically acceptable salt” includes salts of compounds of Formula I and its prodrugs derived from the combination of a compound of this invention and an organic or inorganic acid or base. Suitable acids include acetic acid, adipic acid, benzenesulfonic acid, (+)-7,7-dimethyl-2-oxobicyclo[2.2.1]heptane-1-methanesulfonic acid, citric acid, 1,2-ethanedisulfonic acid, dodecyl sulfonic acid, fumaric acid, glucoheptonic acid, gluconic acid, glucuronic acid, hippuric acid, hydrochloride hemiethanolic acid, HBr, HCl, HI, 2-hydroxyethanesulfonic acid, lactic acid, lactobionic acid, maleic acid, methanesulfonic acid, methylbromide acid, methyl sulfuric acid, 2-naphthalenesulfonic acid, nitric acid, oleic acid, 4,4′-methylenebis[3-hydroxy-2-naphthalenecarboxylic acid], phosphoric acid, polygalacturonic acid, stearic acid, succinic acid, sulfuric acid, sulfosalicylic acid, tannic acid, tartaric acid, terphthalic acid, and p-toluenesulfonic acid.

The terms “patient” and “subject” are used interchangeably, and may include in vitro and in vivo subjects such as cells, tissues, and animals. In this regard, the term “animal” includes birds and mammals. In one embodiment a mammal includes a dog, cat, cow, horse, goat, sheep, pig or human. In one embodiment the animal is a human. In another embodiment the animal is a male. In another embodiment the animal is a female.

The term “increased or enhanced liver specificity” refers to an increase in the liver specificity ratio in animals treated with a compound of the present invention and a control compound. In one embodiment the test compound is a sulfonic acid compound of the present invention and in another embodiment the test compound is a prodrug thereof. In one embodiment the control compound is a sulfur-containing compound of the present invention. In another embodiment the control compound is the corresponding carboxylic acid derivative of the sulfur-containing test compound.

The term “enhanced oral bioavailability” refers to an increase of at least 50% of the absorption of the dose of the parent drug, unless otherwise specified. In an additional aspect the increase in oral bioavailability of the prodrug (compared to the parent drug) is at least 100%, that is a doubling of the absorption. Measurement of oral bioavailability usually refers to measurements of the prodrug, drug, or drug metabolite in blood, plasma, tissues, or urine following oral administration compared to measurements following systemic administration of the compound administered orally.

The terms “treating” or “treatment” of a disease includes a slowing of the progress or development of a disease after onset or actually reversing some or all of the disease affects. Treatment also includes palliative treatment.

The term “preventing” includes a slowing of the progress or development of a disease before onset or precluding onset of a disease.

Compounds of the Invention

In one aspect of the invention, sulfonic acid-containing compounds that are thyroid receptor ligands are provided, pharmaceutically acceptable salts thereof, and prodrugs of these compounds as well as their preparation and uses for preventing and/or treating metabolic diseases such as obesity, NASH, hypercholesterolemia and hyperlipidemia as well as associated conditions such as atherosclerosis, coronary heart disease, impaired glucose tolerance and diabetes. The invention is also related to the liver specific delivery of thyroid receptor ligands and the use of these compounds for the prevention and treatment of diseases responsive to modulation of T3-responsive genes in the liver.

Preferred compounds of the present invention include those of Formula I as shown below.

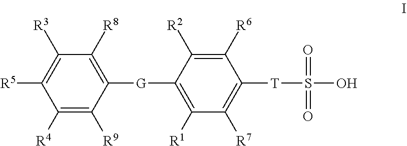

wherein:
G is selected from:

| | | | | |
|---|---|---|---|---|
| —O— | —S— | —Se— | —S(═O)— | —S(═O)₂— |
| —CH₂— | —CHF— | —CF₂— | —C(O)— | —CH(OH)— |
| —CH(C₁-C₄ | —CH(C₁-C₄ | —C(═CH₂)— | —NH— | —N(C₁-C₄ alkyl)- |
| alkyl)- | alkoxy)- | | | |
| —CH₂ linked to | R⁵⁰—R⁵¹ | | | |
| any of the | | | | |
| preceding groups | | | | |

R⁵⁰-R⁵¹ are independently selected from:

| | | |
|---|---|---|
| —O— | —S— | —CH(R⁵³)— |

provisos that at least one R⁵⁰ and R⁵¹ is —CH(R⁵³)—,
and when one of R50 and R51 is
—O— or —S—, then R⁵³ is R⁵⁴

or together R⁵⁰-R⁵¹ form: —C(R⁵²)═C(R⁵²);
R⁵² is selected from:

| | | | |
|---|---|---|---|
| hydrogen | halogen | C₁-C₄ alkyl | C₂-C₄ alkenyl |
| C₂-C₄ alkynyl | C₁-C₄ alkoxy | fluoromethyl | difluoromethyl |
| trifluoromethyl | fluoromethoxy | difluoromethoxy | trifluoromethoxy |
| methylthio | fluoromethylthio | difluoromethylthio | trifluoromethylthio |

R⁵³ is selected from:

| | | | |
|---|---|---|---|
| hydrogen | halogen | hydroxyl | mercapto |
| C₁-C₄ alkyl | C₂-C₄ alkenyl | C₂-C₄ alkynyl | C₁-C₄ alkoxy |
| fluoromethyl | difluoromethyl | trifluoromethyl | fluoromethoxy |
| difluoromethoxy | trifluoromethoxy | methylthio | fluoromethylthio |
| difluoromethylthio | trifluoromethylthio | | |

R⁵⁴ is selected from:

| | | | |
|---|---|---|---|
| hydrogen | halogen | C₁-C₄ alkyl | C₂-C₄ alkenyl |
| C₂-C₄ alkynyl | fluoromethyl | difluoromethyl | trifluoromethyl |

T is selected from:

| | | |
|---|---|---|
| —(CR^{a}₂)ₖ— | —CR^{b}═CR^{b}—(CR^{a}₂)ₙ— | —(CR^{a}₂)ₙ—CR^{b}═CR^{b}— |
| —(CR^{a}₂)—CR^{b}═CR^{b}-(CR^{a}₂)— | —O(CR^{b}₂)(CR^{a}₂)ₙ— | —S(CR^{b}₂)(CR^{a}₂)ₙ— |
| —N(R^{c})(CR^{b}₂)(CR^{a}₂)ₙ— | —N(R^{b})C(O)(CR^{a}₂)ₙ— | —N(R^{b})S(O)2(CR^{a}₂)ₙ— |
| —(CR^{a}₂)ₘC(R^{b})(NR^{b}R^{c})— | —C(O)(CR^{a}₂)ₘ— | —S(O)₂(CR^{a}₂)ₘ— |
| —(Cr^{a}₂)ₘC(O)— | —C(O)N(R^{c})(CR^{b}₂)(CR^{a}₂)ₚ— | —S(O)₂N(R^{c})(CR^{b}₂)(CR^{a}₂)ₚ— |
| —(CR^{b}₂)—O—(CR^{b}₂)—(CR^{a}2)ₚ— | —(CR^{b}₂)—S—(CR^{b}₂)—(CR^{a}₂)ₚ— | —(CR^{b}₂)—N(R^{c})—(CR^{b}₂)(CR^{a}₂)ₚ— |
| —(CR^{a}₂)ₚ—(CR^{b}₂)—O—(CR^{b}₂)— | —(CR^{a}₂)ₚ—(CR^{b}₂)—S—(CR^{b}₂)— | —(CR^{a}₂)ₚ—(CR^{b}₂)N(R^{c})—(CR^{b}₂)— |
| —(CH₂)ₚC(O)N(R^{c})C(R^{b}₂)— | —(CH₂)ₚS(O)₂N(R^{c})C(R^{b}₂)— | —(CR^{a}₂)ₙ—(CR^{b}2)ₚN(R^{c})— |
| —(CR^{a}₂)ₙ—(CR^{b}₂)ₚO— | | |

Each R^{a} is independently selected from:

| | | | | |
|---|---|---|---|---|
| hydrogen | halogen | —OH | —OCF₃ | —OCHF₂ |
| —OCH₂F | —NR^{b}R^{c} | | | |
| optionally | optionally | optionally | optionally | optionally |
| substituted- | substituted | substituted | substituted | substituted |
| C₁-C₄ alkyl | —C₂-C₄ | —C₂-C₄ | —S—C₁- | —O—C₁-C₄ |
| | alkenyl | alkynyl | C₄ alkyl | alkyl |

proviso that when one R^{a} is attached to C through an O, S,
or N atom, then the other R^{a} attached to the
same C is a hydrogen, or attached via a carbon atom

Each R^{b} is independently selected from:

| | |
|---|---|
| hydrogen | optionally substituted —C₁-C₄ alkyl |

Each R^{c} is independently selected from:

| | | | |
|---|---|---|---|
| hydrogen | optionally substituted | optionally substituted | —C(O)H |
| | —C₁-C₄ alkyl | —C(O)—C₁-C₄ | |
| | | alkyl | |

k is an integer from 0-4;
m is an integer from 0-3;
n is an integer from 0-2;
p is an integer from 0-1;
R¹, R², R⁶, and R⁷ are each independently selected from:

| | | | | |
|---|---|---|---|---|
| hydrogen | halogen | —CF₃ | —CHF₂ | —CH₂F |
| —OCF₃ | —OCHF₂ | —OCH₂F | cyano | |
| optionally | optionally | optionally | optionally | optionally |
| substituted —C₁- | substituted —S—C₁- | substituted —C₂- | substituted —C₂- | substituted —O—C₁- |
| C₄ alkyl | C₃ alkyl | C₄ alkenyl | C₄ alkynyl | C₃ alkyl |

proviso that at least one of R¹ and R² is not hydrogen

R³ and R⁴ are each independently selected from:

| | | | |
|---|---|---|---|
| hydrogen | halogen | —CF₃ | —OCF₃ |
| cyano | —OR^{d} | —SR^{d} | —S(O)R^{e} |
| —S(O)₂R^{e} | —S(O)₂NR^{f}R^{g} | —C(O)NR^{f}R^{g} | —C(O)OR^{h} |
| —C(O)R^{e} | —N(R^{b})C(O)R^{e} | —N(R^{b})C(O)NR^{f}R^{g} | —N(R^{b})S(O)₂R^{e} |
| —N(R^{b})S(O)₂NR^{f}R^{g} | —NR^{f}R^{g} | —C≡C(aryl) | —C(R^{b})═C(R^{b})-aryl |
| —C≡C(cycloalkyl) | —C(R^{b})═C(R^{b})— | —C≡C(heterocycloalkyl) | —C(R^{b})═C(R^{b})— |
| | cycloalkyl | | heterocycloalkyl |
| optionally | optionally | optionally substituted | optionally |
| substituted —C₁-C₁₂ | substituted —C₂-C₁₂ | —C₂-C₁₂ alkynyl | substituted |
| alkyl | alkenyl | | —(CR^{a}₂)ₘaryl |
| optionally | optionally | | |
| substituted —(CR^{a}₂)ₘ | substituted —(CR^{a}₂)ₘ | | |
| cycloalkyl | heterocycloalkyl | | |

R⁵ is selected from:

| | | | |
|---|---|---|---|
| —OH | —F | —OC(O)R^{e} | —OC(O)OR^{h} |
| —OC(O)NH(R^{h}) | —NHC(O)OR^{h} | —NHC(O)R^{e} | —NHS(O)R^{e} |
| —NHS(O)₂R^{e} | —NHC(S)NH(R^{h}) | —NHC(O)NH(R^{h}) | optionally |
| | | | substituted —OC₁-C₆ |
| | | | alkyl |

R⁸ and R⁹ are each independently selected from:

| | | | | |
|---|---|---|---|---|
| hydrogen | halogen | hydroxy | —CF₃ | —CHF₂ |
| —CH₂F | —OCF₃ | —OCHF₂ | cyano | —C(O)alkyl |
| —(CR^{a}₂)aryl | —C(O)aryl | —C(O)cycloalkyl | —(CR^{a}₂)cycloalkyl | —C(O)— |
| | | | | heterocycloalkyl |
| —(CR^{a}₂)— | optionally | optionally | optionally | optionally |
| heterocycloalkyl | substituted —C₁- | substituted —S—C₁- | substituted —C₂- | substituted —C₂-C₄ |
| | C₄ alkyl | C₃ alkyl | C₄ alkenyl | alkynyl |
| optionally | | | | |
| substituted | | | | |
| —O—C₁-C₃ alkyl | | | | |

Each R^{d} is independently selected from:

| | | | |
|---|---|---|---|
| —C(O)NR^{f}R^{g} | optionally substituted | optionally substituted | optionally |
| | —C₁-C₁₂ alkyl | —C₂-C₁₂ alkenyl | substituted —C₂-C₁₂ |
| | | | alkynyl |
| optionally | optionally substituted | optionally substituted | |
| substituted —(CR^{b}₂)ₙ | —(CR^{b}₂)ₙcycloalkyl | —(CR^{b}₂)ₙ | |
| aryl | | heterocycloalkyl | |

Each R^{e} is independently selected from:

| | | | |
|---|---|---|---|
| optionally substituted | optionally substituted | optionally | optionally |
| —C₁-C₁₂ alkyl | —C₂-C₁₂ alkenyl | substituted —C₂-C₁₂ | substituted —(CR^{a}₂)ₙ |
| | | alkynyl | aryl |
| optionally substituted | optionally substituted | | |
| —(CR^{a}₂)ₙcycloalkyl | —(CR^{a}₂)ₙ | | |
| | heterocycloalkyl | | |

R^{f} and R^{g} are each independently selected from:

| | | | |
|---|---|---|---|
| hydrogen | optionally substituted | optionally substituted | optionally |
| | —C₁-C₁₂ alkyl | —C₂-C₁₂ alkenyl | substituted —C₂- |
| | | | C₁₂ alkynyl |
| optionally | optionally substituted | optionally substituted | |
| substituted | —(CR^{b}₂)ₙcycloalkyl | —(CR^{b}₂)ₙheterocycloalkyl | |
| —(CR^{b}₂)ₙaryl | | | |

or R^{f} and R^{g} may together form:

an optionally substituted heterocyclic ring of 3-8 atoms containing 0-4
unsaturations, said heterocyclic ring may contain a second heterogroup
within the ring selected from the group consisting of O, NR^{c}, and S
wherein said optionally substituted heterocyclic ring may be substituted
with 0-4 substituents selected from the group consisting of optionally
substituted —C₁-C₄ alkyl, —OR^{b}, oxo, cyano, —CF₃, —CHF₂, —CH₂F,
optionally substituted phenyl, and —C(O)OR^{h}

Each R^{h} is independently selected from:

| | | | |
|---|---|---|---|
| optionally | optionally | optionally | optionally |
| substituted —C₁-C₁₂ | substituted —C₂-C₁₂ | substituted —C₂-C₁₂ | substituted |
| alkyl | alkenyl | alkynyl | —(CR^{b}₂)ₙaryl |
| optionally | optionally | | |
| substituted —(CR^{b}₂)ₙ | substituted —(CR^{b}₂)ₙ | | |
| cycloalkyl | heterocycloalkyl | | |

or R⁶ and T are taken together along with the carbons they are attached to form:

an optionally substituted ring of 5 to 6 atoms with 0-2 unsaturations, not
including the unsaturation on the ring to which R³ and R⁵ are attached,
including 0 to 2 heteroatoms independently selected from —NRⁱ—, —O—,
and —S—
with the proviso that when there are 2 heteroatoms in the ring and both
heteroatoms are different than nitrogen then both heteroatoms have to be
separated by at least one carbon atom; and X is attached to this ring by a
direct bond to a ring carbon, or by —(CR^{a}₂)— or —C(O)— bonded to a ring
carbon or a ring nitrogen

Rⁱ is selected from:

| | | | |
|---|---|---|---|
| hydrogen | —C(O)C₁-C₄ alkyl | —C₁-C₄ alkyl | —C₁-C₄-aryl |

or R¹ and R⁷ are taken together along with the carbons to which they are attached to form:

an optionally substituted ring of 5 to 6 atoms with 0-2 unsaturations, not
including the unsaturation on the ring to which R¹ and R⁷ are attached,
including 0 to 2 heteroatom independently selected froms —NRⁱ—, —O—,
and —S—
with the proviso that when there are 2 heteroatoms in the ring and both
heteroatoms are different than nitrogen then both heteroatoms have to be
separated by at least one carbon atom

or R³ and R⁸ may together along with the carbon atoms to which they are attached to form:

an optionally substituted ring of 5 to 6 atoms with 0-2 unsaturations, not
including the unsaturation on the ring to which R3 and R8 are attached,
including 0 to 2 heteroatoms independently selected from —NRⁱ—, —O—,
and —S—
with the proviso that when there are 2 heteroatoms in the ring and both
heteroatoms are different than nitrogen then both heteroatoms have to be
separated by at least one carbon atom

or R⁸ and G may together along with the carbon atoms to which they are attached to form:

an optionally substituted ring comprising —CH═CH—CH═,
—N═CH—CH═, —CH═N—CH═ or —CH═CH—N═;

R³ and R⁵ are taken together along with the carbons they are attached to form:

an optionally substituted ring of 5 to 6 atoms with 0-2 unsaturations, not
including the unsaturation on the ring to which R³ and R⁵ are attached,
including 0 to 2 heteroatoms independently selected from —NRⁱ—, —O—,
and —S—
with the proviso that when there are 2 heteroatoms in the ring and both
heteroatoms are different than nitrogen then both heteroatoms have to be
separated by at least one carbon atom

and pharmaceutically acceptable salts and prodrugs thereof and pharmaceutically acceptable salts of said prodrugs.

In certain preferred embodiments of Formula I:

G is selected from:

| | | | | |
|---|---|---|---|---|
| —O— | —S— | —Se— | —S(O)— | —S(O)₂— |
| —CH₂— | —CHF— | —CF₂— | —C(O)— | —CH(OH)— |
| —CH(C₁-C₄ alkyl)- | —CH(C₁-C₄ | —C(═CH₂)— | —NH— | —N(C₁-C₄ alkyl)- |
| | alkoxy)- | | | |

T is selected from:

| | | |
|---|---|---|
| —(CR^{a}₂)ₖ— | —CR^{b}═CR^{b}—(CR^{a}₂)ₙ— | —(CR^{a}₂)ₙ—CR^{b}═CR^{b}— |
| —(CR^{a}₂)—CR^{b}═CR^{b}—(CR^{a}₂)— | —O(CR^{b}₂)(CR^{a}₂)ₙ— | —S(CR^{b}₂)(CR^{a}₂)ₙ— |
| —(CR^{a}₂)ₘC(R^{b})(NR^{b}R^{c})— | —C(O)(CR^{a}₂)ₘ— | —S(O)₂(CR^{a}₂)ₘ— |
| —(CR^{a}₂)ₘC(O)— | —C(O)N(R^{c})(CR^{b}₂)(CR^{a}₂)ₚ— | —S(O)₂N(R^{c})(CR^{b}₂)(CR^{a}₂)ₚ— |
| —(CR^{b}₂)—O—(CR^{b}₂)—(CR^{a}₂)ₚ— | —(CR^{b}₂)—S—(CR^{b}₂)—(CR^{a}₂)ₚ— | —(CR^{b}₂)—N(R^{c})—(CR^{b}₂)—(CR^{a}₂)ₚ— |
| —(CR^{a}₂)ₚ—(CR^{b}₂)—O—(CR^{b}₂)— | —(CR^{a}₂)ₚ—(CR^{b}₂)—S—(CR^{b}₂)— | —(CR^{a}₂)ₚ—(CR^{b}₂)—N(R^{c})—(CR^{b}₂)— |
| —(CR^{a}₂)ₘO— | —(CH₂)ₚS(O)₂N(R^{c})C(R^{b}₂)— | —(CR^{a}₂)ₘN(R^{c})— |

Each R^{a} is independently selected from:

| | | | | |
|---|---|---|---|---|
| hydrogen | halogen | —OH | —OCF₃ | —OCHF₂ |
| —OCH₂F | —NR^{b}R^{c} | | | |
| optionally | optionally | optionally | optionally | optionally |
| substituted —C₁- | substituted —C₂- | substituted —C₂- | substituted —S—C₁- | substituted —O—C₁- |
| C₄ alkyl | C₄ alkenyl | C₄ alkynyl | C₄ alkyl | C₄ alkyl |

proviso that when one R^{a} is attached to C through an O, S, or N atom, then the other R^{a} attached
to the same C is a hydrogen, or attached via a carbon atom

Each R^{b} is independently selected from:

| | |
|---|---|
| hydrogen | optionally substituted —C₁-C₄ alkyl |

Each R^{c} is independently selected from:

| | | | |
|---|---|---|---|
| hydrogen | optionally substituted | optionally substituted | —C(O)H |
| | —C₁-C₄ alkyl | —C(O)—C₁-C₄ | |
| | | alkyl | |

k is an integer from 0-4;
m is an integer from 0-3;
n is an integer from 0-2;
p is an integer from 0-1;
R¹, R², R⁶, and R⁷ are each independently selected from:

| | | | | |
|---|---|---|---|---|
| hydrogen | halogen | —CF₃ | —CHF₂ | —CH₂F |
| —OCF₃ | —OCHF₂ | —OCH₂F | cyano | |
| optionally | optionally | optionally | optionally | optionally |
| substituted | substituted | substituted | substituted | substituted |
| —C₁-C₄ | —S—C₁-C₃ | —C₂-C₄ | —C₂-C₄ | —O—C₁-C₃ |
| alkyl | alkyl | alkenyl | alkynyl | alkyl |

proviso that at least one of R¹ and R² is not hydrogen

R³ and R⁴ are each independently selected from:

| | | | |
|---|---|---|---|
| hydrogen | halogen | —CF₃ | —OCF₃ |
| cyano | —OR^{d} | —SR^{d} | —S(O)R^{e} |
| —S(O)₂R^{e} | —S(O)₂NR^{f}R^{g} | —C(O)NR^{f}R^{g} | —C(O)OR^{h}, |
| —C(O)R^{e} | —N(R^{b})C(O)R^{e} | —N(R^{b})C(O)NR^{f}R^{g} | —N(R^{b})S(O)₂R^{e} |
| —N(R^{b})S(O)₂NR^{f}R^{g} | —NR^{f}R^{g} | —C≡C(aryl) | —C(R^{b})═C(R^{b})-aryl |
| —C≡C(cycloalkyl) | —C(R^{b})═C(R^{b})— | —C≡C(heterocycloalkyl) | —C(R^{b})═C(R^{b})— |
| | cycloalkyl | | heterocycloalkyl |
| optionally | optionally | optionally substituted | optionally |
| substituted | substituted —C₂-C₁₂ | —C₂-C₁₂ alkynyl | substituted |
| —C₁-C₁₂ alkyl | alkenyl | | —(CR^{a}₂)ₘaryl |
| optionally | optionally | | |
| substituted | substituted —(CR^{a}₂)ₘ | | |
| —(CR^{a}₂)ₘ cycloalkyl | heterocycloalkyl | | |

R⁵ is selected from:

| | | | |
|---|---|---|---|
| OH | —F | —OC(O)R^{e} | —OC(O)OR^{h} |
| —OC(O)NH(R^{h}) | —NHC(O)OR^{h} | —NHC(O)R^{e} | —NHS(O)R^{e} |
| —NHS(O)₂R^{e} | —NHC(S)NH(R^{h}) | —NHC(O)NH(R^{h}) | optionally |
| | | | substituted |
| | | | —OC₁-C₆ alkyl |

R⁸ and R⁹ are each independently selected from:

| | | | | |
|---|---|---|---|---|
| hydrogen | halogen | hydroxy | —CF₃ | —CHF₂ |
| —CH₂F | —OCF₃ | —OCHF₂ | cyano | —C(O)alkyl |
| —(CR^{a}₂)aryl | —C(O)aryl | —C(O)cycloalkyl | —(CR^{a}₂)cycloalkyl | —C(O)— |
| | | | | heterocycloalkyl |
| —(CR^{a}₂)— | optionally | optionally | optionally | optionally |
| heterocycloalkyl | substituted | substituted | substituted | substituted |
| | —C₁-C₄ | —S—C₁-C₃ | —C₂-C₄ alkenyl | —C₂-C₄ |
| | alkyl | alkyl | | alkynyl |
| optionally | | | | |
| substituted | | | | |
| —O—C₁-C₃ alkyl | | | | |

Each R^{d} is independently selected from:

| | | | |
|---|---|---|---|
| —C(O)NR^{f}R^{g} | optionally substituted | optionally substituted | optionally |
| | —C₁-C₁₂ alkyl | —C₂-C₁₂ alkenyl | substituted |
| | | | —C₂-C₁₂ |
| | | | alkynyl |
| optionally | optionally substituted | optionally substituted | |
| substituted | —(CR^{b}₂)ₙcycloalkyl | —(CR^{b}₂)ₙ | |
| —(CR^{b}₂)ₙ aryl | | heterocycloalkyl | |

Each R^{e} is independently selected from:

| | | | |
|---|---|---|---|
| optionally substituted | optionally substituted | optionally | optionally |
| —C₁-C₁₂ alkyl | —C₂-C₁₂ alkenyl | substituted | substituted |
| | | —C₂-C₁₂ | —(CR^{a}₂)ₙ |
| | | alkynyl | aryl |
| optionally substituted | optionally substituted | | |
| —(CR^{a}₂)ₙcycloalkyl | —(CR^{a}₂)ₙ | | |
| | heterocycloalkyl | | |

R^{f} and R^{g} are each independently selected from:

| | | | |
|---|---|---|---|
| hydrogen | optionally | optionally | optionally |
| | substituted | substituted | substituted |
| | —C₁-C₁₂ alkyl | —C₂-C₁₂ alkenyl | —C₂-C₁₂ |
| | | | alkynyl |
| optionally | optionally | optionally substituted | |
| substituted | substituted | —(CR^{b}₂)ₙheterocycloalkyl | |
| —(CR^{b}₂)ₙaryl | —(CR^{b}₂)ₙcycloalkyl | | |

or R^{f} and R^{g} may together form:

an optionally substituted heterocyclic ring of 3-8 atoms containing 0-4 unsaturations, said
heterocyclic ring may contain a second heterogroup within the ring selected from the group
consisting of O, NR^{c}, and S
wherein said optionally substituted heterocyclic ring may be substituted with 0-4 substituents
selected from the group consisting of optionally substituted —C₁-C₄ alkyl, —OR^{b}, oxo, cyano,
—CF₃, —CHF₂, —CH₂F, optionally substituted phenyl, and —C(O)OR^{h}

Each R^{h} is independently selected from:

| | | | |
|---|---|---|---|
| optionally | optionally | optionally | optionally |
| substituted | substituted | substituted | substituted |
| —C₁-C₁₂ | —C₂-C₁₂ | —C₂-C₁₂ | —(CR^{b}₂)ₙaryl |
| alkyl | alkenyl | alkynyl | |
| optionally | optionally | | |
| substituted —(CR^{b}₂)ₙ | substituted —(CR^{b}₂)ₙ | | |
| cycloalkyl | heterocycloalkyl | | |

or R⁶ and T are taken together along with the carbons they are attached to form:

an optionally substituted ring of 5 to 6 atoms with 0-2 unsaturations, not
including the unsaturation on the ring to which R³ and R⁵ are attached, including
0 to 2 heteroatoms independently selected from —NRⁱ—, —O—, and —S—
with the proviso that when there are 2 heteroatoms in the ring and both hetero-
atoms are different than nitrogen then both heteroatoms have to be separated by
at least one carbon atom; and X is attached to this ring by a direct bond to a ring
carbon, or by —(CR^{a}₂)— or —C(O)— bonded to a ring carbon or a ring
nitrogen

Rⁱ is selected from:

| | | | |
|---|---|---|---|
| hydrogen | —C(O)C₁-C₄ alkyl | —C₁-C₄ alkyl | —C₁-C₄—aryl |

or R¹ and R⁷ are taken together along with the carbons to which they are attached to form:

an optionally substituted ring of 5 to 6 atoms with 0-2 unsaturations, not
including the unsaturation on the ring to which R¹ and R⁷ are attached, including
0 to 2 heteroatoms independently selected from —NRⁱ—, —O—, and —S—
with the proviso that when there are 2 heteroatoms in the ring
and both heteroatoms are different than nitrogen then both heteroatoms
have to be separated by at least one carbon atom

or R³ and R⁸ may together along with the carbon atoms to which they are attached to form:

an optionally substituted ring of 5 to 6 atoms with 0-2 unsaturations, not
including the unsaturation on the ring to which R³ and R⁸ are attached, including
0 to 2 heteroatoms independently selected from —NRⁱ—, —O—, and —S—
with the proviso that when there are 2 heteroatoms in the ring
and both heteroatoms are different than nitrogen then both heteroatoms
have to be separated by at least one carbon atom

or R⁸ and G may together along with the carbon atoms to which they are attached to form:

an optionally substituted ring comprising —CH═CH—CH═,
—N═CH-CH═, —CH═N—CH═ or —CH═CH-N═;

and pharmaceutically acceptable salts and prodrugs thereof and pharmaceutically acceptable salts of said prodrugs.

With reference is Formula I, in certain preferred embodiments, R⁶, R⁷, R⁸, and R⁹ may each be hydrogen. Further, in certain embodiments, R¹, R², R³, and R⁴ may each be selected from C₁ to C₄ alkyls. In other embodiments, R⁵ is preferably —OH. In addition, in certain preferred embodiments, G is preferably —O— or —CH₂—. In other embodiments, T is preferably —(CR^{a}₂)ₖ— or —O(CR^{b}₂)(CR^{a}₂)ₚ—.

In other embodiments, preferred compounds of Formula I include compounds of Formula IA shown below:

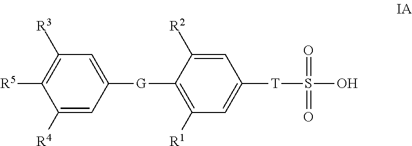

wherein:
G is selected from the group consisting of:

| | | | |
|---|---|---|---|
| —O —, | —S— | —Se— | —S(O)— |
| —S(O)₂— | —CH₂— | —CHF— | —CF₂— |
| —C(O)— | —CH(OH)— | —NH— | —N(C₁-C₄ alkyl)— |
| CH₂ linked to any of | R⁵⁰—R⁵¹ | | |
| the preceding groups | | | |

R⁵⁰-R⁵¹ together are —C(R⁵²)═C(R⁵²)— or;
R⁵⁰ and R⁵¹ are independently selected from:

| | | |
|---|---|---|
| —O— | —S— | —CH(R⁵³)— |

with the provisos that at least one R⁵⁰ and R⁵¹ is —CH(R⁵³)—, and when one of R⁵¹ is O or S, then R⁵³ is R⁵⁴

R⁵² is selected from:

| | | | |
|---|---|---|---|
| hydrogen | halogen | C₁-C₄ alkyl | C₂-C₄ alkenyl |
| C₂-C₄ alkynyl | C₁-C₄ alkoxy | fluoromethyl | difluoromethyl |
| trifluoromethyl | fluoromethoxy | difluoromethoxy | trifluoromethoxy |
| methylthio | fluoromethylthio | difluoromethylthio | trifluoromethylthio |

R⁵³ is selected from:

| | | | |
|---|---|---|---|
| hydrogen | halogen | hydroxyl | mercapto |
| C ₗ-C₄ alkyl | C₂-C₄ alkenyl | C₂-C₄ alkynyl | C₁-C₄ alkoxy |
| fluoromethyl | difluoromethyl | trifluoromethyl | fluoromethoxy |
| difluoromethoxy | trifluoromethoxy | methylthio | fluoromethylthio |
| difluoromethylthio | trifluoromethylthio | | |

R⁵⁴ is selected from:

| | | | |
|---|---|---|---|
| hydrogen | halogen | C₁-C₄ alkyl | C₂-C₄ alkenyl |
| C₂-C₄ alkynyl | fluoromethyl | difluoromethyl | trifluoromethyl |

T is selected from:

| | | |
|---|---|---|
| —(CR^{a}₂)ₖ— | —CR^{b}═CR^{b}—(CR^{a}₂)ₙ— | —(CR^{a}₂)ₙ—CR^{b}═CR^{b}— |
| —(CR^{a}₂)—CR^{b}═CR^{b}(CR^{a}₂)— | —O(CR^{b}₂)(CR^{a}₂)ₙ— | —S(CR^{b}₂)(CR^{a}₂)ₙ— |
| —N(R^{c})(CR^{b}₂)(CR^{a}₂)ₙ— | —N(R^{b})C(O)(CR^{a}₂)ₙ— | —N(R^{b})S(O)₂(CR^{a}₂)ₙ— |
| —(CR^{a}₂)ₘC(R^{b})(NR^{b}R^{c})— | —C(O)(CR^{a}₂)ₙ— | —S(O)₂(CR^{a}₂)ₘ— |
| —(CR^{a}₂)ₘC(O)— | —C(O)N(R^{c})(CR^{b}₂)(CR^{a}₂)ₚ— | —S(O)₂N(R^{c})(CR^{b}₂)(CR^{a}₂)ₚ— |
| —(CR^{b}₂)—O—(CR^{b}₂)—(CR^{a}₂)ₚ— | —(CR^{b}₂)—S—(CR^{b}₂)—(CR^{a}₂)ₚ— | —(CR^{b}₂)—N(R^{c})—(CR^{b}₂)—(CR^{a}₂)ₚ— |
| —(CR^{a}₂)ₚ—(CR^{b}₂)—O—(CR^{b}₂)— | —(CR^{a}₂)p—(CR^{b}₂)—S—(CR^{b}₂)— | —(CR^{a}₂)ₚ—(CR^{b}₂)—N(R^{c})—(CR^{b}₂)— |
| —(CH₂)ₚC(O)N(R^{c})C(R^{b}₂)— | —(CH₂)ₚS(O)₂N(R^{c})C(R^{b}₂)— | —(CR^{a}₂)ₙ—(CR^{b}₂)ₚN(R^{c})— |
| —(CR^{a}₂)ₙ—(CR^{b}₂)ₚO— | | |

k is an integer from 0-4;
m is an integer from 0-3;
n is an integer from 0-2;
p is an integer from 0-1;
Each R^{a} is independently selected from:

| | | | |
|---|---|---|---|
| hydrogen | —OCF₃ | —OCHF₂ | —OCH₂F |
| —NR^{b}R^{c} | halogen | —OH | optionally |
| | | | substituted |
| | | | —C₁-C₄ alkyl |
| optionally | optionally | optionally | optionally |
| substituted | substituted | substituted | substituted |
| —O—C₁-C₄ alkyl | —S—C₁-C₄ alkyl | —C₂-C₄ alkenyl | —C₂-C₄ alkynyl |

with the proviso that when one R^{a} is attached to C through an O, S, or N atom, then the other Rₐ attached to the same C is a hydrogen, or attached via a carbon atom

Each R^{b} is independently selected from:

| | |
|---|---|
| hydrogen | optionally substituted —C₁-C₄ alkyl |

Each R^{c} is independently selected from:

| | | | |
|---|---|---|---|
| hydrogen | —C(O)H | optionally substituted | optionally substituted |
| | | —C₁-C₄ alkyl | —C(O)—C₁-C₄ alkyl |

R¹ and R² are each independently selected from:

| | | | |
|---|---|---|---|
| halogen | —CF₃ | —CHF₂ | —CH₂F |
| —OCF₃ | —OCHF₂ | —OCH₂F | cyano |
| optionally | optionally | optionally | optionally |
| substituted | substituted | substituted | substituted |
| —C₁-C₄ alkyl | —S—C₁-C₃ alkyl | —C₂-C₄ alkenyl | —C₂-C₄ alkynyl |
| optionally | | | |
| substituted | | | |
| —O—C₁-C₃ alkyl | | | |

R³ and R⁴ are each independently selected from:

| | | | |
|---|---|---|---|
| hydrogen | halogen | —CF₃ | —CHF₂ |
| —CH₂F | —OCF₃ | —OCHF₂ | —OCH₂F |
| cyano | —C(R^{b})═C(R^{b})-aryl | —C(R^{b})═C(R^{b})-cycloalkyl | —C(R^{b})═C(R^{b})— |
| | | | heterocycloalkyl |
| —C≡C(aryl) | —C≡C(cycloalkyl) | —C≡C(heterocycloalkyl) | —(CR^{a}₂)ₙ(CR^{b}₂)NR^{f}R^{g} |
| —OR^{d} | —SR^{d} | —S(O)Re | —S(O)₂R^{e} |
| —S(O)₂NR^{f}R^{g} | —C(O)NR^{f}R^{g} | —C(O)ORh | —C(O)R^{e} |
| —N(R^{b})C(O)R^{e} | —N(R^{b})C(O)NR^{f}R^{g} | —N(R^{b})S(O)₂R^{e} | —N(R^{b})S(O)₂NR^{f}R^{g} |
| —NR^{f}R^{g} | optionally | optionally substituted | optionally substituted |
| | substituted —C₁-C₁₂ | —C₂-C₁₂ alkenyl | —C₂-C₁₂ alkynyl |
| | alkyl | | |
| optionally | optionally | optionally substituted | |
| substituted | substituted —(CR^{a}₂)ₘ | —(CR^{a}₂)ₘheterocycloalkyl | |
| —(CR^{a}₂)ₘ aryl | cycloalkyl | | |

R⁵ is selected from:

| | | | |
|---|---|---|---|
| —OH | —F | —OC(O)R^{e} | —OC(O)OR^{h,} |
| —NHC(O)OR^{h} | —OC(O)NH(R^{h}) | —NHC(O)R^{e} | —NHS(O)R^{e} |
| —NHS(O)2R^{e} | —NHC(S)NH(R^{h}) | —NHC(O)NH(R^{h}) | optionally |
| | | | substituted |
| | | | —OC₁-C₆ |
| | | | alkyl |

R³ and R⁵ are taken together along with the carbons they are attached to form:

an optionally substituted ring of 5 to 6 atoms with 0-2 unsaturations,
not including the unsaturation on the ring to which R³ and R⁵ are attached,
including 0 to 2 heteroatoms independently selected from —NRⁱ—,
—O—, and —S— with the proviso that when there are 2 heteroatoms
in the ring and both heteroatoms are different than nitrogen then both
heteroatoms have to be separated by at least one carbon atom

Each R^{d} is selected from:

| | | | |
|---|---|---|---|
| —C(O)NR^{f}R^{g} | optionally | optionally | optionally |
| | substituted | substituted | substituted |
| | —C₁-C₁₂ | —C₂-C₁₂ | —C₂-C₁₂ |
| | alkyl | alkenyl | alkynyl |
| optionally | optionally | optionally | |
| substituted | substituted | substituted | |
| —(CR^{b}₂)ₙ | —(CR^{b}₂)ₙ | —(CR^{b}₂)ₙ | |
| aryl | cycloalkyl | heterocycloalkyl | |

Each R^{e} is selected from:

| | | | |
|---|---|---|---|
| optionally substituted | optionally substituted | optionally | optionally |
| —C₁-C₁₂ alkyl | —C₂—C₁₂ alkenyl | substituted | substituted |
| | | —C₂—C₁₂ | —(CR^{a}₂)ₙaryl |
| optionally substituted | optionally substituted | alkyny | |
| —(CR^{a}₂)ₙcycloalkyl | —(CR^{a}₂)ₙ | | |
| | heterocycloalkyl | | |

R^{f} and R^{g} are each independently selected from:

| | | | |
|---|---|---|---|
| hydrogen | optionally substituted- | optionally | optionally |
| | C₁-C₁₂ alkyl | substituted- | substituted- |
| | | C₂-C₁₂ alkenyl | C₂-C₁₂ |
| | | | alkynyl |
| optionally | optionally substituted- | optionally | |
| substituted- | (CR^{b}₂)ₙ cycloalkyl | substituted- | |
| (CR^{b}₂)ₙ aryl | | (CR^{b}₂)ₙ | |
| | | heterocycloalkyl | |

R^{f} and R^{g} may together form:

an optionally substituted heterocyclic ring of 3-8 atoms containing
0-4 unsaturations, which may contain a second heterogroup selected
from the group of O, NR^{c}, and S
wherein said optionally substituted heterocyclic ring may be substituted
with 0-4 substituents selected from the group consisting of optionally
substituted-C₁-C₄ alkyl, —OR^{b}, oxo, cyano, —CF₃,
—CHF₂, —CH₂F, optionally substituted phenyl, and —C(O)OR^{h}

Each R^{h} is selected from:

| | | | |
|---|---|---|---|
| optionally | optionally | optionally | optionally |
| substituted- | substituted- | substituted- | substituted- |
| C₁-C₁₂ alkyl | C₂-C₁₂ alkenyl | C₂-C₁₂ alkynyl | (CR^{b}₂)ₙ aryl |
| optionally | optionally | | |
| substituted- | substituted- | | |
| (CR^{b}₂)ₙ cycloalkyl | (CR^{b}₂)ₙ | | |
| | heterocycloalkyl | | |

Rⁱ is selected from:

| | | | |
|---|---|---|---|
| hydrogen | —C(O)C₁-C₄ alkyl | —C₁-C₄ alkyl | —C₁-C₄-aryl |

and pharmaceutically acceptable salts and prodrugs thereof and pharmaceutically acceptable salts of said prodrugs.

With reference is Formula IA, in certain preferred embodiments, R¹, R², R³, and R⁴ may each be selected from C₁ to C₄ alkyls. In other embodiments, R⁵ is preferably —OH. In addition, in certain preferred embodiments, G is preferably —O— or —CH₂—. In other embodiments, T is preferably —(CR^{a}₂)ₖ— or —O(CR^{b}₂)(CR^{a}₂)ₚ—

In yet other embodiments, preferred compounds of Formula I include compounds of Formula IB, shown below.

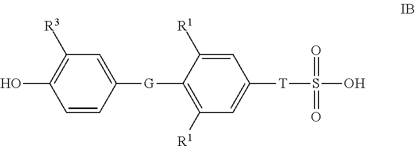

wherein:
G is selected from:

| | |
|---|---|
| —O— | —CH₂ |

T is selected from:

| | | |
|---|---|---|
| —(CR^{a}₂)ₙ— | —O(CR^{b}₂)(CR^{a}₂)ₚ— | —S(CR^{b}₂)(CR^{a}₂)ₚ— |
| —N(R^{c})(CR^{b}₂)(CR^{a}₂)ₚ— | —(CR^{b}₂)ₙN(R^{c})— | —(CR^{b}₂)ₙO— |

n is an integer from 0-2;
p is an integer from 0-1;
Each R^{a} is independently selected from:

| | | | |
|---|---|---|---|
| hydrogen | halogen | —OH | —OCF₃ |
| —OCHF₂ | —OCH₂F | —NR^{b}R^{c} | optionally |
| | | | substituted- |
| | | | C₁-C₄ alkyl |
| optionally | optionally substituted- | optionally | optionally |
| substituted- | S—C₁-C₄ alkyl | substituted- | substituted- |
| O—C₁-C₄ alkyl | | C₂-C₄ alkenyl | C₂-C₄ alkynyl |

with the proviso that when one R^{a} is attached to C through an O, S, or N atom, then the other R^{a} attached to the same C is a hydrogen, or attached via a carbon atom

Each R^{b} is independently selected from:

| | |
|---|---|
| hydrogen | optionally substituted-C₁-C₄ alkyl |

Each R^{c} is independently selected from:

| | | | |
|---|---|---|---|
| hydrogen | —C(O)H | optionally | optionally |
| | | substituted- | substituted- |
| | | C₁-C₄ alkyl | C(O)—C₁-C₄ alkyl |

R¹ is selected from:

| | | | |
|---|---|---|---|
| halogen | —CF₃ | cyano | optionally substituted- |
| | | | C₁-C₄ alkyl |

R³ is selected from:

| | | | |
|---|---|---|---|
| halogen | —CF₃ | —CHF₂ | —CH₂F |
| —OCF₃ | —OCHF₂ | —OCH₂F | cyano |
| —C(R^{b})═C(R^{b})-aryl | —C(R^{b})═C(R^{b})- | C(R^{b})═C(R^{b})- | —C≡C(aryl) |
| | cycloalkyl | heterocycloalkyl | |
| —C≡C(cycloalkyl) | —C≡C | —(CR^{a}₂)ₙ(CR^{b}₂)NR^{f}R^{g} | —OR^{d} |
| | (heterocycloalkyl) | | |
| —SR^{d} | —S(O)R^{e} | —S(O)₂R^{e} | —S(O)₂NR^{f}R^{g}, |
| —C(O)NR^{f}R^{g} | —C(O)OR^{h} | —C(O)R^{e} | —N(R^{b})C(O)R^{e} |
| —N(R^{b})C(O)NR^{f}R^{g} | —N(R^{b})S(O)₂R^{e} | —N(R^{b})S(O)₂NR^{f}R^{g} | —NR^{f}R^{g} |
| optionally | optionally | optionally substituted- | optionally |
| substituted-C₁-C₁₂ | substituted-C₂-C₁₂ | C₂-C₁₂ alkynyl | substituted-(CR^{a}₂)ₘ |
| alkyl | alkenyl | | aryl |
| optionally | optionally | | |
| substituted-(CR^{a}₂)ₘ | substituted-(CR^{a}₂)ₘ | | |
| cycloalkyl | heterocycloalkyl | | |

Each R^{d} is independently selected from:

| | | | |
|---|---|---|---|
| —C(O)NR^{f}R^{g} | optionally substituted | optionally | optionally |
| | alkyl | substituted- | substituted- |
| | | C₁-C₁₂ alkenyl | C₂-C₁₂ |
| | | | alkynyl |
| optionally | optionally substituted | optionally | |
| substituted- | (CR^{b}₂)ₙ | substituted- | |
| (CR^{b}₂)ₙ aryl | cycloalkyl | (CR^{b}₂)ₙ | |
| | | heterocycloalkyl | |

Each R^{e} is independently selected from:

| | | | |
|---|---|---|---|
| optionally substituted- | optionally | optionally | optionally |
| C₁-C₁₂ alkyl | substituted- | substituted- | substituted- |
| | C₂-C₁₂ alkenyl | C₂-C₁₂ alkynyl | (CR^{a}₂)ₙaryl |
| optionally substituted- | optionally | | |
| (CR^{a}₂)ₙ cycloalkyl | substituted- | | |
| | (CR^{a}₂)ₙ | | |
| | heterocycloalkyl | | |

R^{f} and R^{g} are each independently selected from:

| | | | |
|---|---|---|---|
| hydrogen | optionally substituted- | optionally | optionally |
| | C₁-C₁₂ alkyl | substituted- | substituted- |
| | | C₂-C₁₂ alkenyl | C₂-C₁₂ alkynyl |
| optionally | optionally substituted- | optionally | |
| substituted- | (CR^{b}₂)ₙ cycloalkyl | substituted- | |
| (CR^{b}₂)ₙ aryl | | (CR^{b}₂)ₙ | |
| | | heterocycloalkyl | |

R^{f} and R^{g} may together form:

an optionally substituted heterocyclic ring of 3-8 atoms containing 0-4
unsaturations, which may contain a second heterogroup selected from
the group of O, NR^{c}, and S
wherein said optionally substituted heterocyclic ring may be substituted
with 0-4 substituents selected from the group consisting of optionally
substituted-C₁-C₄ alkyl, —OR^{b}, oxo, cyano, —CF₃,
—CHF₂, —CH₂F, optionally substituted phenyl, and —C(O)OR^{h}

Each R^{h} is selected from:

| | | | |
|---|---|---|---|
| optionally substituted | optionally substituted | optionally | optionally |
| —C₁-C₁₂ alkyl | —C₂-C₁₂ alkenyl | substituted | substituted |
| | | —C₂-C₁₂ | —(CR^{b}₂)ₙaryl |
| | | alkynyl | |
| optionally substituted | optionally substituted | | |
| —(CR^{b}₂)ₙcycloalkyl | —(CR^{b}₂)ₙhetero- | | |
| | cycloalkyl | | |

and pharmaceutically acceptable salts and prodrugs thereof and pharmaceutically acceptable salts of said prodrugs.

With reference is Formula IB, in certain preferred embodiments, R¹ and R³ may each be selected from C₁ to C₄ alkyls. In other embodiments, T is preferably —(CR^{a}₂)ₙ— or —O(CR^{b}₂)(CR^{a}₂)ₚ—.

In certain embodiments, with reference to the compounds of the invention, when G is selected from —O—, —S—, S(O)—, —S(O)₂—, —CH₂—, —NH—, or —N(C₁-C₄ alkyl)-; R³ and R⁵ may not be taken together along with the carbons they are attached to form a ring structure.

In certain embodiments, with reference to the compounds of the invention, when G is O; R⁵ is selected from —(CH₂)₁₋₃-aryl or —(CH₂)₁₋₃-heteroaryl; R⁶ and T may not be taken together to form the group —(CH₂)₃—.

In other embodiments, with reference to the compounds of the invention, when G is —CH₂— linked to any of the group selected from —O—, —S—, —Se—, S(O)—, —S(O)₂—, —NH—, or —N(C₁-C₄ alkyl)-; T may not be selected from —(CR^{a}₂)ₖ—, —C(O)(CR^{a}₂)ₘ—, —O(CH₂)ₘ—, —S(CH₂)ₘ—, or —N(Rc)(CH₂)ₘ—.

In yet other embodiments, to the extent relevant to the disclosed compounds of the invention, when G is —C(R⁵²)═C(R⁵²)—; R³ and R⁴ may not be selected from —NH₂, —NH—S(O)₂R^{e}, or —NH—C(O)R^{e}, wherein R⁵² is selected from hydrogen, halogen, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio and thiotrifluoromethyl.

In yet other embodiments, with reference to the compounds of the invention, when G is —O—, R⁵ may not be selected from —NHS(O)₂R^{e}, —NHS(O)R^{e}, —NHC(S)NH(R^{h}), —NHC(O)OR^{h}, or —NHC(O)R^{e}.

In yet other embodiments, with reference to the compounds of the invention, when G is —CH₂—O—; T may not be —(CH₂)₁₋₂(CHR^{a})—, wherein R^{a} is selected from halogens, —OH, SH, NH2 and —NH(C₁₋₄).

In yet other embodiments, to the extent relevant to the disclosed compounds of the invention, when G is —O—, —S—, —Se—, —S(═O)₂—, —CH₂—, —C(O)—, —NH—; R¹ and R² are independently chosen from the group consisting of hydrogen, halogen, —C₁-C₄ alkyl; R⁸ and R⁹ are each independently selected from hydrogen, halogen and C₁₋₄alkyl; R⁶ and R⁷ are each independently selected from hydrogen, halogen O—C₁₋₃alkyl, hydroxy, cyano and C₁₋₄alkyl; R³ is —C(O)NR²⁵R²⁶, —CH₂—NR²⁵R²⁶, —NR²⁵—C(O)R²⁶, —OR²⁷, R²⁸, or

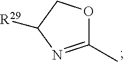

R⁴ is hydrogen, halogen, cyano or alkyl; and R⁵ is —OH; wherein R²⁵ and R²⁶ are each independently selected from the group consisting of hydrogen, aryl, heteroaryl, alkyl, cycloalkyl, aralkyl or heteroaralkyl, R²⁷ is aryl, heteroaryl, alkyl, aralkyl, or heteroaralkyl, R²⁸ is aryl, heteroaryl, or cycloalkyl, and R²⁹ is hydrogen, aryl, heteroaryl, alkyl, aralkyl, heteroaralkyl; then T may not be —(CH₂)₀₋₄— or —(CH₂)ₚ—C(O)N(R^{c})(CR^{b}₂)—.

In yet another embodiment, to the extent relevant to the disclosed compounds of the invention, when G is —O—; R⁵ is —OH; R⁶, R⁷, R⁸, R⁹ are hydrogen; T is —(CH₂)ₖ—; and R⁴ is not hydrogen; then R³ may not be selected from: a substituted R²⁸—C₂-C₃ alkyl or a substituted R²⁸—C₂-C₃ alkenyl, wherein R²⁸ is aryl, heteroaryl, or cycloalkyl.

In yet another embodiment, with reference to the compounds of the invention, when G is —O—, R¹, R² and R³ are halogen, and T is a bond, then R⁵ may not be OH.

As recognized by one of skill in the art, certain compounds of the invention may include at least one chiral center, and as such may exist as racemic mixtures or as enantiomerically pure compositions. As used herein, “enantiomerically pure” refers to compositions consisting substantially of a single isomer, preferably consisting of 90%, 92%, 95%, 98%, 99%, or 100% of a single isomer.

For the purposes of this invention, where one or more functionalities or substituents are incorporated into a compound of the invention, including preferred embodiments, each functionality or substituent appearing at any location within the disclosed compounds may be independently selected, and as appropriate, independently substituted. Further, where a more generic substituent is set forth for any position in the molecules of the present invention, it is understood that the generic substituent may be replaced with more specific substituents, and the resulting molecules are within the scope of the molecules of the present invention.

Preferred compounds of the invention include the following.

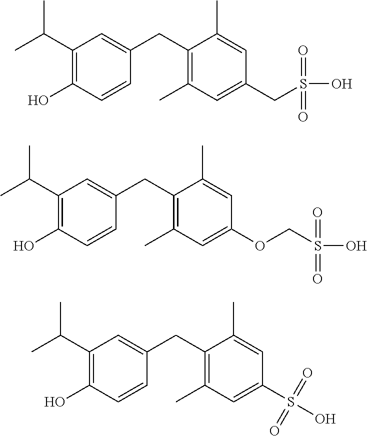

The above compounds are listed only to provide examples that may be used in the methods of the invention. Based upon the instant disclosure, the skilled artisan would recognize other compounds intended to be included within the scope of the presently claimed invention that would be useful in the methods recited herein.

In one aspect, the sulfonic acid-containing compounds, pharmaceutically acceptable salts and prodrugs thereof, and pharmaceutically acceptable salts of the prodrugs used in these methods bind to at least one thyroid hormone receptor with an Ki of ≦100 nM relative to T3, or ≦90 nM, ≦80 nM, ≦70 nM, ≦60 nM, ≦50 nM, ≦40 nM, ≦30 nM, ≦20 nM, ≦10 nM, ≦50 nM, ≦1 nM, ≦0.5 nM. Thyroid hormone receptor binding is readily determined using assays described in the literature. For example, nuclear extracts from animal livers can be prepared according to the methods described by Yokoyama et al. (J. Med. Chem. 38:695-707 (1995)). Binding assays can also be performed using purified thyroid hormone receptors. For example, using the methods used by Chiellini et al. (Bioorg. Med. Chem. 10:333-346 (2002)), competition ligand binding affinities are determined using ¹²⁵I-T3 and the human thyroid receptors TRα1 and TRβ1. The latter methods advantageously enable determination of thyroid receptor selectivity. Methods described herein may be used to determine the binding of compounds of this invention.

In another aspect, the sulfonic acid-containing compounds, pharmaceutically acceptable salts and prodrugs thereof, and pharmaceutically acceptable salts of the prodrugs used in these methods cause at least a 50%, 2 fold, 3 fold, 4 fold, 6 fold or 8 fold increase or decrease in the expression of one or more thyroid hormone-responsive genes. Changes in gene expression can be detected in cells or in vivo.

Changes in gene expression in vivo require either the sulfonic acid of the invention to be taken up by the tissue following administration or for the prodrug remain intact after administration long enough to distribute to the target organ and cell. Following distribution to the cell, enzymes responsible for cleaving the prodrug must act on the prodrug and convert it to the sulfonic acid. The compound must then be able to be transported to the nucleus. If a portion of the compound is excreted from the cell it must be retransported back across the cellular membrane and nuclear membrane. The prodrugs of the present invention that are activated in the liver and excreted by the liver as sulfonic acid compounds are retransported back across the cellular and nuclear membrane and into the nucleus. Despite being excreted from the liver and having to be retransported into the nucleus and despite having reduced potency in vivo, the sulfonic acid-containing compounds and their prodrugs led to surprisingly potent biological activity. This surprisingly high biological activity is attributed to the ability of the compounds of the present invention to modulate genes known to be regulated by T3. For example, mGPDH increased >1.5-fold in the liver of an animal administered a 1 mg/kg dose of the drug.

Also provided are compounds that selectively distribute to the liver. In one embodiment, the compounds have at least 10 fold, 25 fold, 50 fold, 75 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold 6000 fold, 7000 fold, 8000 fold, 9000 fold, 10,000 fold, 20,000 fold, 30,000 fold, 40,000 fold or 50,000 fold greater selectivity. In one embodiment the selectivity for the liver is compared to the heart. In another embodiment the selectivity for the liver is compared to the pituitary. In another embodiment the selectivity for the liver is compared to the kidney.

Also provided are sulfonic acid-containing T3 mimetics or prodrug thereof that have improved liver selectivity as compared to a corresponding compound where the sulfur-containing group is replaced with a carboxylic acid, but wherein the corresponding compound is otherwise identical. In one embodiment, the sulfonic acid-containing compound (or prodrug thereof) has at least 10 fold, 25 fold, 50 fold, 75 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold 6000 fold, 7000 fold, 8000 fold, 9000 fold, 10,000 fold, 20,000 fold, 30,000 fold, 40,000 fold or 50,000 fold greater selectivity for the liver as compared to the corresponding carboxylic acid compound. In one embodiment the liver selectivity is relative to the heart. In another embodiment the liver selectivity is relative to the kidney. In another embodiment the liver selectivity is relative to the pituitary.

Also provided are sulfonic acid-containing T3 mimetics or prodrug thereof that have a decreased Ki as compared to a corresponding compound where the sulfur-containing group is replaced with a carboxylic acid, but wherein the corresponding compound is otherwise identical. In one embodiment, the sulfonic acid-containing compound has at least 2 fold, 5 fold, 7 fold, 10 fold, 25 fold, or 50 fold lower Ki than the corresponding carboxylic acid derivative compound (wherein Ki is measured relative to T3). In another embodiment, the Ki of the sulfonic acid-containing compound is ≦150 nM ≦100 nM, ≦90 nM, ≦80 nM, ≦70 nM, ≦60 nM, ≦50 nM, ≦40 nM, ≦30 nM, relative to T3. For purposes of clarity, it is noted that binding affinity increases as the numerical value of Ki decreases, i.e., there is an inverse relationship between Ki and binding affinity. In another embodiment the sulfonic acid-containing compound has the same Ki as the corresponding carboxylic acid derivative. In another embodiment the sulfonic acid-containing compound has a greater Ki than the corresponding carboxylic acid derivative.

Also provided are compounds of the present invention that bind at least one thyroid hormone receptor with an Ki of ≦100 nM, ≦90 nM, ≦80 nM, ≦70 nM, ≦60 nM, ≦50 nM, ≦40 nM, ≦30 nM, ≦20 nM, ≦10 nM, ≦50 nM, ≦1 nM, or ≦0.5 nM relative to T3. In one embodiment said thyroid hormone receptor is TRα. In one embodiment said thyroid hormone receptor is TRβ. Also provided are compounds that bind at least one thyroid hormone receptor with an Ki of ≧100 nM, ≧90 nM, ≧80 nM, ≧70 nM, ≧60 nM, ≧50 nM, ≧40 nM, ≧30 nM, ≧20 nM, ≧10 nM, ≧50 nM, ≧1 nM, or ≧0.5 nM relative to T3, but in each case ≦150 nM. In one embodiment said thyroid hormone receptor is TRα. In one embodiment said thyroid hormone receptor is TRβ. In one embodiment said thyroid hormone receptor is TRα1. In one embodiment said thyroid hormone receptor is TRβ1. In one embodiment said thyroid hormone receptor is TRα2. In one embodiment said thyroid hormone receptor is TRβ2.

Preparation of Compounds of the Invention

The compounds in this invention may be prepared by the processes described herein in the following general schemes and examples, as well as relevant published literature procedures that are used by those skilled in the art. It should be understood that the following schemes are provided solely for the purpose of illustration and do not limit the invention which is defined by the claims.

All stereoisomers of the compounds of the instant invention are contemplated, either in admixture or in pure or substantially pure form. The compounds of the present invention can have stereogenic centers. Consequently, the compounds can exist in enantiomeric or diastereomeric forms or in mixture thereof. The processes for preparation can utilize racemates, enantiomers or diastereomers as starting materials. When enantiomeric or diastereomeric products are prepared, they can be separated by conventional methods for example, chromatographic or fractional crystallization.

### Construction of Diaryl Group

Again, the compounds of the invention may generally be prepared according to processes known in the art, including processes similar to those described in WO 2006/128055 (the contents of which are herein incorporated by reference in their entirety), modified as described herein and as recognized by those in the art.

### Introduction of a Sulfonic Acid Group

The introduction of a sulfonic acid group can generally be accomplished according to known methods. Compounds of the invention wherein T is O(CR^{b}₂)(CR^{a}₂)₀₋₂—, —N(R^{c})(CR^{b}₂)(CR^{a}₂)₀₋₂—, —S(CR^{b}₂)(CR^{a}₂)₀₋₂—, —(CR^{b}₂)O(CR^{b}₂)(CR^{a}₂)₀₋₁—, —(CR^{b}₂)N(R^{c}) (CR^{b}₂)(CR^{a}₂)₀₋₁—, —(CR^{b}₂)S(CR^{b}₂)(CR^{a}₂)₀₋₁—, can be prepared by coupling a phenol, thiophenol, aniline, alcohol, amine or mercaptan with a haloalkyl sulfonate such as Br(CR^{a}₂)₁₋₃S(O)₂Na or Cl(CR^{b}₂)₁₋₃S(O)₂Na, in the presence of a base such as NaOH or t-BuOK, (Bioorg Med. Chem. Lett. 7:1583 (1997); J. Chem. Res. Synap. 720 (1999); Heterocycl. Chem 331-337 (1974). Generally, sodium haloalkyl sulfonates are prepared from dihaloalkanes by treatment with aq Na₂SO₃ (sodium sulfite) to give the corresponding monosodium alkylsulfonates. Compounds of the invention wherein T is —S(O)₁₋₂(CR^{a}₂)₁₋₃—, or —(CR^{a}₂)S(O)₁₋₂(CR^{a}₂)ₚ— can be prepared from the corresponding sulfide as above followed by oxidation to the corresponding sulfoxide or sulfone with an oxidizing agent such as a peracid or sodium chlorite.

Compounds of the invention wherein T is —C(O)N(R^{c})(CR^{b}₂)(CR^{a}₂)₀₋₁— or —S(O)₂N(R^{c})(CR^{b}₂)(CR^{a}₂)₀₋₁— can be prepared by coupling an acid (M=COOH, S(O)₂OH) with HN(R^{c})(CR^{b}₂)(CR^{a}₂)₀₋₁S(O)₂OH in the presence of DCC or EDCI, HOBt, according to the known methods (for example, J. Org. Chem. 42:2019 (1977)

For compounds of the invention wherein T is —(CR^{a}₂)ₖ— the sulfonic acid group can be introduced by a number of known methods. For example, the substitution reaction of a benzyl (or) alkyl bromide/iodide with sodium sulfite. (Bioorg Med. Chem. Lett. 17:3322 (2007)), or ammonium sulfite (Bull. Chem. Soc. Jpn. 649 (1987)). Benzyl/alkyl halides are prepared according to known methods starting from the corresponding alcohols and treatment with PBr₃ (or) PPh₃/I₂ to give corresponding benzyl/alkyl halides.

For compounds of the invention wherein T is a bond, the sulfonic acid group can be introduced by using methods and conditions well-known in the art, via oxidation of the corresponding thiophenol or trialkylsilylthiophenol. For example the hydrogen peroxide/acetic acid mediated oxidation of a thiophenol (J. Org. Chem., 24:1892 (1959)) or KNO₃/SO₂Cl₂ mediated oxidation of a trialkylsilylthiophenol (Tet. Lett., 44:7821 (2003)) or thiophenol (Chem. Lett., 21:1483 (1992)). Trialkylsilylthiophenols are synthesized from the corresponding phenol via palladium coupling of the phenol derivative aryl trifluoromethylsulfonate with trialkylsilyl thiolate (Tetrahedron Lett. 37:4523 (1996)). The silyl group can then be removed with TBAF to give the corresponding thiophenol.

For compounds of the invention wherein T is —(CR^{a}₂)₀₋₂(CR^{b}₂)₀₋₁N(R^{c})— or —(CR^{a}₂)₀₋₂(CR^{b}₂)₀₋₁O—, the sulfamic acid or sulfate group can be introduced by coupling of an amine with catechol sulfate in the presence of a base such as KOH and followed by hydrolysis to give the corresponding sulfamic acid (J. Org. Chem. 45:5371 (1980) or reaction of a phenol with SO₃/Py (J. Med. Chem. 45:598 (2002))

For compounds of the invention wherein T is —C(O)(CR^{a}₂)— or —(CR^{a}₂)C(O)(CR^{a}₂)—, the aceto-sulfonic-acid group can be introduced by reacting alpha bromo ketones with ammonium sulfite (Recl. Trav. Chim. Pays-Bas 47, 166 (1928) or sodium sulfite, Chem. Ber. 75:1348 (1942).

For compounds of the invention wherein T is —N(R^{b})C(O)(CR^{a}₂)(CR^{a}₂)ₚ—, —N(R^{b})S(O)₂(CR^{a}₂)(CR^{a}₂)ₚ— the carboxamide or sulfonamide group can be introduced by reacting an aniline with the corresponding sulfonylacetic acid with DCC (Bioorg. Med. Chem. Lett. 8:289 (1998)) or an alkyl disulfonate such as methylene disulfonate (Recl. Trav. Chim. Pays-Bas; 54; 208 (1935))

In certain preferred embodiments, compounds of the invention may be resolved to enantiomerically pure compositions or synthesized as enantiomerically pure compositions using any method known in art. By way of example, compounds of the invention may be resolved by direct crystallization of enantiomer mixtures, by diastereomer salt formation of enantiomers, by the formation and separation of diasteriomers or by enzymatic resolution of a racemic mixture.

These and other reaction methodologies may be useful in preparing the compounds of the invention, as recognized by one of skill in the art. Various modifications to the above schemes and procedures will be apparent to one of skill in the art, and the invention is not limited specifically by the method of preparing the compounds of the invention.

Methods of the Invention

In another aspect of the invention, methods are provided. In preferred embodiments, the methods of the invention comprise administering a therapeutically effective amount of at least one compound of the invention, e.g., a compound of Formula I. Relative activity of the compounds of the invention may be determined by any method known in the art, including the assay described herein.

In one aspect, the sulfonic acid-containing thyromimetics and their prodrugs and salts are useful in preventing or treating arteriosclerosis by modulating levels of atherogenic proteins, e.g., Lp(a), apoAI, apoAII, LDL, HDL. Clinically overt hypothyroidism is associated with accelerated and premature coronary atherosclerosis and subclinical hypothyroidism is considered a condition with an increased risk for these diseases (Vanhaelst et al. and Bastenie et al., Lancet 2 (1967)).

T3 and T3 mimetics modulate atherogenic proteins in a manner that could prove beneficial for patients at risk to develop atherosclerosis or patients with atherosclerosis or diseases associated with atherosclerosis. T3 and T3 mimetics are known to decrease Lp(a) levels, e.g., in the monkey, with 3,5-dichloro-4-[4-hydroxy-3-(1-methylethyl)phenoxy]benzeneacetic acid (Grover et al., Proc. Natl. Acad. Sci. U.S.A. 100:10067-10072 (2003)). In human hepatoma cells, the T3 mimetic CGS23425 ([[4-[4-hydroxy-3-(1-methylethyl)phenoxy]-3,5-dimethylphenyl]amino]oxo acetic acid) increased apoAI expression via thyroid hormone receptor activation (Taylor et al., Mol. Pharm. 52.542-547 (1997)).

Thus in one aspect, the sulfonic acid-containing thyromimetics, their salts and prodrugs can be used to treat or prevent atherosclerosis, coronary heart disease and heart failure because such compounds are expected to distribute to the liver and modulate the expression and production of atherogenic proteins.

In another aspect, the sulfonic acid-containing thyromimetics and their prodrugs and salts are useful for preventing and/or treating metabolic diseases such as obesity, hypercholesterolemia and hyperlipidemia and conditions such as atherosclerosis, coronary heart disease, heart failure, nephrotic syndrome, and chronic renal failure without affecting thyroid function, thyroid production of circulating iodinated thyronines such as T3 and T4, and/or the ratio of T3 to T4. Compounds previously reported that contain a carboxylic acid moiety, e.g., GC-1 ([4-[[4-hydroxy-3-(1-methylethyl)phenyl]methyl]-3,5-dimethylphenoxy]acetic acid) (Trost et al., Endocrinology 141:3057-3064 (2000)) and 3,5-Dichloro-4-[4-hydroxy-3-(1-methylethyl)phenoxy]benzeneacetic acid (Grover et al., Proc. Natl. Acad. Sci. U.S.A. 100:10067-10072 (2003)) report that these TRβ-selective compounds dose-dependently lower cholesterol and TSH levels. Effects on cholesterol and TSH occur at the same dose or at doses stated to be not pharmacologically different (e.g., 2-fold).

Particularly useful T3 mimetics in these methods would minimize effects on thyroid function, thyroid production of circulating iodinated thyronines such as T3 and T4, and/or the ratio of T3 to T4. Unlike prior T3 mimetics, the compounds or the present invention distribute more readily to the liver and result in pharmacological effects at doses that do not adversely affect thyroid function, thyroid production of circulating iodinated thyronines such as T3 and T4, and/or the ratio of T3 to T4. In one embodiment the compounds of the present invention have a therapeutic index, defined as the difference between the dose at which a significant effect is observed for a use disclosed herein, e.g., lowering cholesterol, and the dose at which a significant decrease in T3 or significant decrease in T4, or significant change in the ratio of T3 to T4 is observed, is at least 50 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold or at least 10000 fold. In one embodiment, rather than a significant amount, the amount of change in T3 or T4 is a decrease selected from at least 5%, 10%, 15%, 20%, 25% or at least 30% of circulating levels.

In one embodiment, the sulfonic acid-containing thyromimetics and their prodrugs and salts are useful for significantly lowering cholesterol levels without having a significant effect on TSH levels. In another embodiment, the compounds of the present invention significantly lower cholesterol levels without lowering TSH levels by more than 30%, 25%, 20%, 15%, 10%, or 5%.

Side effects associated with TH-based therapies limit their use for treating obese patients and according to the Physician's Desk Reference (PDR) T3 is now contraindicated for patients with obesity. 3,5-dichloro-4-[4-hydroxy-3-(1-methylethyl)phenoxy]benzeneacetic acid and other T3 mimetics are reported to result in weight loss in animals, e.g., rodent models and monkeys. Weight loss from these compounds may arise from their effects on the liver as well as peripheral tissues. TH is known to have a multitude of effects outside of the liver that could result in increased metabolism and weight loss. TH plays an important role in the development and function of brown and white adipose tissue. TH can induce WAT differentiation, proliferation and intracellular lipid accumulation. TH induces lipogenic genes in WAT such as glucose-6-phosphate dehydrogenase, fatty acid synthase and spot-14. TH also regulates lipolysis in fat to produce weight loss in a coordinated manner, i.e., lipolysis in fat to free fatty acids followed by free fatty acid utilization in tissues, e.g., liver, muscle and heart.

Weight loss through administration of liver-specific T3 analogues requires that the increased oxygen consumption in the liver resulting from T3 is sufficient to result in net whole body energy expenditure. The liver's contribution to energy expenditure is estimated to be 22% based on oxygen consumption measurements. (Hsu, A et al. Am. J. Clin. Nutr. 77(6):1506-11 (2003)). Thus, the compounds of the present invention may be used to maintain or reduce weight in an animal.

Mitochondria are the fuel source for all cellular respiration. The synthesis of new mitochondria is a complex process which requires over 1000 genes (Goffart et al., Exp. Physiol. 88(1):33-40 (2003)). The mechanisms which control mitochondrial biogenesis are not well defined, but are known to include exercise (Jones et al., Am. J. Physiol. Endocrinol. Metab. 284(1):E96-101 (2003)), overexpression of PGC-1 (Lehman et al., J. Clin. Invest. 106(7):847-56 (2000)) or AMP activated protein kinase (Bergeron et al., Am. J. Physiol. Endocrinol. Metab. 281(6):E1340-6 (2001)). An increase in mitochondrial density leads to a greater rate of energy expenditure. Thyroid hormone has been shown to play a key role in mitochondrial biogenesis by increasing expression of nuclear respiratory factor-1 and PGC-1 (Weitzel et al., Exp. Physiol. 88(1):121-8 (2003)).

Compounds which increase the expression of NRF-1 and/or PGC-1 could lead to an increase in mitochondrial density within a cell. Such an increase would cause the cell to have a higher rate of energy expenditure. Methods to analyze NRF-1 and PGC-1 include immunoblotting with specific antibodies, or analysis of mRNA levels. Compounds that caused increases in NRF-1 or PGC-1 would therefore lead to a greater energy expenditure. Even small increases in energy expenditure over long periods of time (weeks to years) could cause a decrease in weight under isocaloric circumstances. Further methods for assessing mitochondrial biogenesis include the analysis of mitochondrial proteins such as cytochrome c and cytochrome c oxidase, either by immunoblotting or analysis of mRNA levels. Mitochondrial density can also be measured by counting the number of mitochondria in electron micrographs.

In one aspect, sulfonic acid-containing thyromimetics and their prodrugs and salts may be used to cause weight loss or prevent weight gain without side effects. It may be advantageous to use compounds that result in high liver specificity (Examples F and G). In one aspect, compounds that result in increased levels of genes associated with oxygen consumption, e.g., GPDH (Example B), are particularly useful in weight loss and controlling weight gain. In another aspect, compounds that show weight loss at doses that do not affect cardiac function, e.g., heart rate, force of systolic contraction, duration of diastolic relaxation, vascular tone, or heart weight, may be particularly useful in weight loss and controlling weight gain. In a further aspect, compounds that cause weight loss without affecting thyroid function, thyroid production of circulating iodinated thyronines such as T3 and T4, and/or the ratio of T3 to T4 are particularly useful.

Besides their use in obesity and weight control, sulfonic acid-containing thyromimetics and their prodrugs and salts may be used to treat diabetes and related conditions like impaired glucose tolerance, insulin resistance and hyperinsulinemia.

Patients with type 2 diabetes “T2DMs” exhibit chronic high blood glucose levels. High fasting blood glucose in T2DMs is related to the overproduction of glucose by a pathway in the liver known as the gluconeogenesis pathway. Throughput in this pathway is controlled in part by enzymes in the pathway such as PEPCK, fructose 1,6-bisphosphatase and glucose 6-phosphatase as well as by hormones such as insulin, which can influence the expression and activities of these enzymes. T3 is known to worsen diabetes. While the reason T3 worsens diabetes is not known, T3's effect on increasing the gene expression of PEPCK and glucose-6-phosphatase may be the cause of increased glucose levels. T3 is known to increase lipolysis of triglyceride pools in fat and to increase circulating levels of free fatty acids. (K. S. Park, et al., Metabolism 48(10):1318-21 (1999)) T3's effect on free fatty acid levels may also be responsible for the negative effect on diabetes because high free fatty acid levels enhance flux through the gluconeogenesis pathway.

Compounds of this invention, while they mimic T3, result in preferential activation of liver T3 genes, are not expected to increase lipolysis in peripheral tissues which is expected to avoid the T3-induced higher circulating levels of free fatty acids and their effects on increasing gluconeogenesis flux and decreasing insulin sensitivity. Increased hepatic insulin sensitivity will decrease PEPCK and glucose 6-phosphatase gene expression thus reducing gluconeogenesis. TR activation in the liver should also decrease liver fat content, which in turn is expected to improve diabetes and steatohepatitis (e.g., NASH), thus providing another use for the compounds of the present invention. A decrease in liver fat content is associated with increased hepatic insulin sensitivity (Shulman, 2000) and accordingly should improve glycemic control in type 2 diabetics through decreased glucose production and enhanced glucose uptake. The overall effect on the patient will be better glycemic control, thus providing another use for the compounds of the present invention.

TH also stimulates GLUT-4 transporter expression in skeletal muscle which produces concomitant increases in basal glucose uptake. Studies in obese, insulin-resistant Zucker rats showed that TH therapy induces GLUT-4 expression in skeletal muscle and total amelioration of the hyperinsulinemia, although plasma glucose levels were moderately elevated (Torrance et al. Endocrinology 138:1204 (1997)). Thus another embodiment of the present invention relates to the use of compounds of the present invention to prevent or treat hyperinsulinemia.

TH therapy results in increased energy expenditure. Increased energy expenditure can result in increased weight loss, which in turn can result in improved glycemic control. Diet and exercise are often used initially to treat diabetics. Exercise and weight loss increase insulin sensitivity and improve glycemia. Thus, further uses of the compounds of the present invention include increasing energy expenditure, increasing insulin sensitivity and improving glycemia.

In one aspect, the sulfonic acid-containing compounds of the present invention are useful for increasing levels of genes associated with gluconeogenesis (Example B). In another aspect, the compounds of the present invention are useful for decreasing hepatic glycogen levels. Further, compounds of the present invention result in amelioration of hyperinsulinemia and/or decreased glucose levels in diabetic animal models at doses that do not affect cardiac function, e.g., heart rate, force of systolic contraction, duration of diastolic relaxation, vascular tone, or heart weight. In a further aspect, compounds of the present invention result in amelioration of hyperinsulinemia and/or decreased glucose levels in diabetic animal models at doses that do not affect thyroid function, thyroid production of circulating iodinated thyronines such as T3 and T4, and/or the ratio of T3 to T4.

As discussed above, the previous use of T3 and T3 mimetics to treat metabolic diseases have been limited by the deleterious side-effects on the heart. Previous attempts to overcome this limitation have focused on selectively targeting the liver over the heart using T3 mimetics that selectively bind TRβ over TRα. Because the heart expresses mainly TRα, previous investigators have attempted to increase the therapeutic index of T3 mimetics by increasing the selectively of the compounds for TRβ which is expressed in the liver. Previous attempts have not focused on T3 mimetics that selectively distribute to the liver over the heart or at least have not been successful. Thus, rather than selecting for a particular tissue or organ, previous work has been directed to discovering T3 mimetics that act selectively at the receptor level after the drug is non-selectively distributed to both heart and liver tissue. It was therefore unexpected when the present Inventors discovered that the sulfonic acid-compounds of the present invention selectively distributed to the liver over the heart. The selective distribution to the liver over the heart was also found with prodrugs, that although were processed in the liver, were excreted from the liver into the blood stream as active sulfonic acid compounds. Thus the compounds of the present invention are able to selectively target the liver and thereby increase the therapeutic index as compared to T3 and T3 mimetics containing a carboxylic acid. The compounds of the present invention can therefore be dosed at levels that are effective in treating metabolic and other disorders where the liver is the drug target without significantly negatively affecting heart function.

Because of the selectivity of the sulfonic acid-containing compounds of the present invention for the liver over the heart, it is not necessary for the compound to have greater selectivity for TRβ over TRα, although this may be desired. In fact, surprisingly some of the compounds of the present invention selectively bind TRα over TRβ and are highly effective for the uses disclosed herein without having the negative side-effects normally associated with TRα selective compounds. Thus, included as an embodiment of the present invention are compounds of the invention that selectively bind TRβ over TRα by at least 5 fold, 10 fold, 20 fold, 30 fold, 40 fold, 50 fold, 60 fold, 70 fold, 80 fold, 90 fold, 100 fold, 200 fold, 300 fold, 400 fold or at least 500 fold, and compounds of the invention that selectively bind TRα over TRβ by at least 5 fold, 10 fold, 20 fold, 30 fold, 40 fold, 50 fold, 60 fold, 70 fold, 80 fold, 90 fold, 100 fold, 200 fold, 300 fold, 400 fold or at least 500 fold.

Changes in the therapeutic index are readily determined using assays and methods well described in the literature. Genes in extrahepatic tissues are monitored using methods well understood by those skilled in the art. Assays include using cDNA microarray analysis of tissues isolated from treated animals. The sensitivity of the heart to T3 makes analysis of T3-responsive genes in the heart as well as the functional consequences of these changes on cardiac properties one further strategy for evaluating the therapeutic index of the compounds of the present invention. Cardiac genes measured include mGPDH and myosin heavy and light chain. One method of measuring the effects of T3 mimetics on the heart is by the use of assays that measure T3 mediated myosin heavy chain gene transcription in the heart.

In one embodiment the compounds of the present invention have a therapeutic index, defined as the difference between the dose at which a significant effect is observed for a use disclosed herein, e.g., lowering cholesterol, and the dose at which a significant effect on a property or function, as disclosed herein (e.g., heart rate), is observed, is at least 50 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold or at least 10000 fold. Examples of said use disclosed herein includes but is not limited to reducing lipid levels, increasing the ratio of HDL to LDL or apoAI to LDL, reducing weight or preventing weight gain, maintaining or improving glycemic control, lowering blood glucose levels, increasing mitochondrial biogenesis, increasing expression of PGC-1, AMP activated protein kinase or nuclear respiratory factor, inhibiting hepatic gluconeogenesis or for the treatment or prevention of a disease or disorder selected from the group consisting of atherosclerosis, hypercholesterolemia, hyperlipidemia, obesity, NASH, NAFLD, nephrotic syndrome, chronic renal failure, insulin resistance, diabetes, metabolic syndrome X, impaired glucose tolerance, hyperlipidemia, coronary heart disease, thyroid disease, thyroid cancer, depression, glaucoma, cardiac arrhythmias, heart failure, and osteoporosis. Examples wherein the property or function is a cardiac property/function include but are not limited to cardiac hypertrophy (heart weight to body weight ratio), heart rate, and various hemodynamic parameters, including systolic and diastolic arterial pressure, end systolic left ventricular pressure and maximal speeds of contraction and relaxation.

A variety of methods are described that provide a means for evaluating the functional consequences of T3-cardiac action, including measurement of cardiac hypertrophy (heart weight to body weight ratio), heart rate, and various hemodynamic parameters, including systolic and diastolic arterial pressure, end-systolic left ventricular pressure and maximal speeds of contraction and relaxation using methods described by Trost et al., (Endocrinology 141:3057-64 (2000)). Other methods are also available to assess the therapeutic index including effects on muscle wasting and bone density.

The therapeutic index is determined by administering to animals a wide range of doses and determining the minimal dose capable of inducing a response in the liver relative to the dose capable of inducing a response in the heart.

In vivo assays include but are not limited to treating animals with sulfonic acid-containing compounds of the invention or a prodrug thereof and monitoring the expression of T3-responsive genes in the liver or the functional consequences of changes of T3-responsive genes.

In one aspect, compounds useful in the novel methods bind to thyroid receptors and produce changes in the expression of two or more hepatic genes. Animals used for testing compounds useful in the methods include normal rats and mice, animals made hypothyroid using methods well described in the literature, including thyroid hormone receptor knockout mice (e.g., TRα^{-/-} such as those used in Grover et al., 2003), or animals exhibiting high cholesterol (e.g., high cholesterol fed rat or hamster), obesity and/or diabetes (e.g., fa/fa rat, Zucker diabetic fatty rat, ob/ob mice, db/db mice, high fat fed rodent). (Liureau et al., Biochem. Pharmacol. 35(10):1691-6 (1986); Trost et al., Endocrinology 141(9):3057-64 (2000); and Grover et al., 2003).

The drug or prodrug may be administered by a variety of routes including by bolus injection, oral, and continuous infusion. By way of example, animals may be treated for 1-28 days and the liver, heart and blood are isolated. Changes in gene transcription relative to vehicle treated animals and T3-treated animals determined using northern blot analysis, RNAase protection or reverse-transcription and subsequent PCR. While methods are available for monitoring changes in thousands of hepatic genes, only a small number need to be monitored to demonstrate the biological effect of compounds in this invention. Typically, genes such as spot-14, FAS, mGPDH, CPT-1, and LDL receptor may be monitored. Changes of >1.5 fold in two or more genes may be considered proof that the compound modulates T3-responsive genes in vivo. Alternative methods for measuring changes in gene transcription include monitoring the activity or expression level of the protein encoded by the gene. For instance, in cases where the genes encode enzyme activities (e.g., FAS, mGPDH), direct measurements of enzyme activity in appropriately extracted liver tissue can be made using standard enzymological techniques. In cases where the genes encode receptor functions (e.g., the LDL receptor), ligand binding studies or antibody-based assays (e.g., Western blots) can be performed to quantify the number of receptors expressed. Depending on the gene, TR agonists may either increase or decrease enzyme activity or increase or decrease receptor binding or number.

The functional consequences of changing the expression levels of hepatic genes responsive to T3 is many-fold and readily demonstrated using assays well described in the literature. Administering sulfonic acid-containing compounds that bind to a TR to animals can result in changes in lipids, including hepatic and/or plasma cholesterol levels; changes in lipoprotein levels including LDL-cholesterol, lipoprotein a (Lp(a)); changes in hepatic glycogen levels; and changes in energy expenditure as measured by changes in oxygen consumption and in some cases animal weight. For example, the effect on cholesterol may be determined using cholesterol fed animals such as normal rats and hamsters, or TRα^{-/-} knockout mice. Cholesterol may be measured using standard tests. Hepatic glycogen levels may be determined from livers isolated from treated animals. Changes in energy expenditure may be monitored by measuring changes in oxygen consumption (MV_{O}_{₂}). Varieties of methods are well described in the literature and include measurement in the whole animal using Oxymax chambers (U.S. Pat. No. 6,441,015). Livers from treated rats can also be evaluated (Fernandez et al., Toxicol. Lett. 69(2):205-10 (1993)) as well as isolated mitochondria from liver (Carreras et al., Am. J. Physiol. Heart Circ. Physiol. 281(6):H2282-8 (2001)). Hepatocytes from treated rats can also be evaluated (Ismail-Beigi F et al., J Gen Physiol. 73(3):369-83 (1979)).

Sulfonic acid-containing compounds that bind to a TR modulate expression of certain genes in the liver resulting in effects on lipids (e.g., cholesterol), glucose, lipoproteins, and triglycerides. Such compounds can lower cholesterol levels which is useful in the treatment of patients with hypercholesterolemia. Such compounds can lower levels of lipoproteins such as Lp(a) or LDL and are useful in preventing or treating atherosclerosis and heart disease in patients. Such compounds can raise levels of lipoproteins such as apoAI or HDL and are useful in preventing or treating atherosclerosis and heart disease in patients. Such compounds can cause a reduction in weight. Such compounds can lower glucose levels in patients with diabetes.

Also provided are methods of reducing plasma lipid levels in an animal, the method comprising the step of administering to a patient an amount of a compound of the invention. In one embodiment said compound is an active form. In another embodiment said compound is a prodrug. In another embodiment said compound of the invention comprises a stereocenter, is enantiomerically enriched or diastereomerically enriched, or a stereoisomer covered later. In another embodiment said compound is administered as a racemic mixture. In another embodiment said compound is administered as an enantiomerically enriched mixture. In another embodiment said compound is a administered as a diastereomerically enriched mixture. In still another embodiment said compound is administered as an individual stereoisomer.

Also provided are methods of reducing plasma lipid levels in an animal wherein the lipid is cholesterol. In one embodiment said methods of reducing cholesterol results in a lowering of total cholesterol. In one embodiment said methods of reducing cholesterol results in a reduction of high density lipoprotein (HDL). In one embodiment said methods of reducing cholesterol results in a reduction of low density lipoprotein (LDL). In one embodiment said methods of reducing cholesterol results in a reduction of very low density lipoprotein (VLDL). In another embodiment said LDL is reduced to a greater extent than said HDL. In another embodiment said VLDL is reduced to a greater extent than said HDL. In another embodiment said VLDL is reduced to a greater extent than said LDL.

In one embodiment of the method of reducing lipids, the lipid is triglycerides. In one embodiment said lipid is liver triglycerides. In another embodiment said lipid is in the form of a lipoprotein. In another embodiment said lipoprotein is Lp(a). In another embodiment said lipoprotein is apoAII. Also provided are methods of increasing the ratio of HDL to LDL, HDL to VLDL, LDL to VLDL, apoAI to LDL or apoAI to VLDL in an animal.

Also provided are methods of treating hyperlipidemia or hypercholesterolemia in an animal, Also provided are methods of preventing or treating atherosclerosis in an animal. Also provided are methods of reducing fat content in the liver or of preventing or treating fatty liver/steatosis, NASH or NAFLD in an animal. Also provided are methods of preventing or treating nephrotic syndrome or chronic renal failure in an animal. Also provided are methods of reducing weight or preventing weight gain in an animal. Also provided are methods of preventing or treating obesity in an animal. Also provided are methods of preventing or treating coronary heart disease in an animal.

Also provided are methods of maintaining or improving glycemic control in an animal being treated with a T3 mimetic. Also provided are methods of lowering blood glucose levels in an animal. Also provided are methods of preventing or treating diabetes, insulin resistance, metabolic syndrome X or impaired glucose tolerance in an animal. Also provided are methods of preventing or treating altered energy expenditure in an animal. Also provided are methods of preventing or treating a liver disease responsive to modulation of T3-responsive genes in an animal. Also provided are methods of preventing or treating thyroid disease, thyroid cancer, depression, glaucoma, cardiac arrhythmias, heart failure, or osteoporosis in an animal. Also provided are methods of increasing mitochondrial biogenesis in an animal. Also provided are methods of increasing expression of PGC-1, AMP activated protein kinase or nuclear respiratory factor in an animal. Also provided are methods of inhibiting hepatic gluconeogenesis in an animal

In all methods described above, the methods generally comprise the step of administering to a patient in need thereof, such as an animal subject including a human subject, an effective amount of a compound of the invention. In one embodiment said compound is an active form. In another embodiment said compound is a prodrug. In another embodiment said compound of the invention comprises a stereocenter. In another embodiment said compound is administered as a racemic mixture. In another embodiment said compound is administered as an enantiomerically enriched mixture. In another embodiment said compound is a administered as a diastereomeric mixture. In still another embodiment said compound is administered as an individual stereoisomer.

Without intending to be limited by theory, it is believed that the methods of the present invention act through a combination of mechanisms. The liver is a major target organ of thyroid hormone with an estimated 8% of the hepatic genes regulated by thyroid hormone. Quantitative fluorescent-labeled cDNA microarray hybridization was used to identify thyroid-responsive genes in the liver as shown in Table 1 below (Feng et al., Mol. Endocrinol. 14:947-955 (2000)). Hepatic RNA from T3-treated and hypothyroid mice were used in the study. Thyroid hormone treatment affected the expression of 55 genes from the 2225 different mouse genes sampled with 14 increasing >2-fold and 41 decreasing >60%.

TABLE 1
List of Hepatic Genes Regulated by T3 Determined
by cDNA Microarray Analyses

| | | | |
|---|---|---|---|
| Function | | Accession | |
| Clone ID | Genes | No. | Fold |

Carbohydrate and fatty acid metabolism, and insulin action

| | | | |
|---|---|---|---|
| 580906 | Spot 14 gene | X95279 | 8.8 |
| 523120 | Glucose-6-phosphatase | U00445 | 3.8 |
| 615159 | Carbonyl reductase (Cbr1) | U31966 | 3.3 |
| 571409 | Insulin-like growth factor binding | X81579 | 3.0 |
| | protein 1 precursor | | |
| 481636 | Fatty acid transport protein (FATP) | U15976 | 1.8 |
| 550993 | Cyp4a-10 | X69296 | 0.3 |
| 583329 | PHAS-II | U75530 | 0.3 |
| 616283 | Serine/threonine kinase (Akt2) | U22445 | 0.3 |
| 583333 | Putative transcription factor of the | X17500 | 0.3 |
| | insulin gene | | |
| 533177 | Nuclear-encoded mitochondrial | L42996 | 0.2 |
| | acyltransferase | | |
| 608607 | Glycerophosphate dehydrogenase | J02655 | 0.3 |

Cell proliferation, Replication

| | | | |
|---|---|---|---|
| 614275 | B61 | U26188 | 2.3 |
| 597868 | Bcl-3 | M90397 | 2.5 |
| 493127 | Kinesin-like protein (Kip1p) | AF131865 | 2.0 |
| 582689 | Chromodomain-helicase-DNA binding | P40201 | 0.4 |
| | protein CHD-1 | | |
| 524471 | NfiB1-protein (exon 1-12) | Y07685 | 0.3 |
| 516208 | Putative ATP-dependent RNA | J04847 | 0.3 |
| | helicase PL10 | | |
| 558121 | Murine vik5variant in the kinase | S53216 | 0.1 |
| 573247 | C11 protein | X81624 | 0.3 |
| 522108 | Thymic stromal stimulating factor | D43804 | 0.3 |
| 613942 | Ubiquitin-activating enzyme E1 X | D10576 | 0.3 |

Signal transduction

| | | | |
|---|---|---|---|
| 573046 | β-2 Adrenergic receptor | X15643 | 3.4 |
| 583258 | Protein kinase C inhibitor (mPKCl) | U60001 | 2.1 |
| 616040 | Inhibitory G protein of adenylate | M13963 | 0.3 |
| | cyclase, α chain | | |
| 583353 | Terminal deoxynucleotidyltransferase | 04123 | 0.3 |
| 550956 | Rho-associated, coiled-coil forming | U58513 | 0.2 |
| | protein kinase p160 | | |
| 582973 | Protein kinase C, Θ type | AB011812 | 0.3 |
| 442989 | Protein kinase ζ | M94632 | 0.5 |
| 607870 | Lamin A | D13181 | 0.3 |

Glycoprotein synthesis

| | | | |
|---|---|---|---|
| 375144 | α-2,3-Sialyltransferase | D28941 | 0.3 |
| 481883 | β-Galactoside α 2,6-sialyltransferase | D16106 | 0.3 |

Cellular immunity

| | | | |
|---|---|---|---|
| 615872 | T-complex protein 1, d subunit | P80315 | 0.3 |
| 618426 | H-2 class I histocompatibility antigen | Q61147 | 0.3 |
| 614012 | FK506-binding protein (FKBP65) | L07063 | 0.3 |
| 604923 | FK506-binding protein (FKBP23) | AF040252 | 0.2 |

Cytoskeletal protein

| | | | |
|---|---|---|---|
| 374030 | Myosin binding protein H (MyBP-H) | U68267 | 2.2 |
| 613905 | AM2 receptor | X67469 | 0.3 |
| 616518 | Cytoskeletal β-actin | X03672 | 0.3 |
| 614948 | Actin, α cardiac | M15501 | 0.3 |
| 607364 | Skeletal muscle actin | M12866 | 0.3 |
| 597566 | Capping protein a-subunit | G565961 | 0.3 |
| 483226 | Actin, γ-enteric smooth muscle | M26689 | 0.3 |

Others

| | | | |
|---|---|---|---|
| 552837 | Major urinary protein 2 precursor | M27608 | 3.9 |
| 521118 | β-Globin | AB020013 | 2.3 |
| 493218 | α-Globin | L75940 | 2.7 |
| 585883 | Putative SH3-containing protein SH3P12 | AF078667 | 0.3 |
| 615239 | Membrane-type matrix metalloproteinase | X83536 | 0.2 |
| 402408 | ecel (endothelin-converting enzyme) | W78610 | 0.2 |
| 635768 | α-Adaptin | P17426 | 0.3 |
| 634827 | Glucose regulated protein 78 | D78645 | 0.3 |
| 616189 | Lupus la protein homolog | L00993 | 0.3 |
| 588337 | EST | AI646753 | 0.4 |
| 335579 | Virus-like (VL30) retrotransposon BVL-1 | X17124 | 0.3 |
| 557037 | TGN38B | D50032 | 0.3 |
| 597390 | Mitochondrial genome | L07096 | 0.4 |
| 616563 | Arylsulfatase A | X73230 | 0.3 |

Genes reported to be affected by thyroid hormone are identified using a variety of techniques include microarray analysis. Studies have identified genes that are affected by T3 and T3 mimetics that are important in metabolic diseases.

T3-responsive genes in the liver include genes affecting lipogenesis, including spot 14, fatty acid transport protein, malic enzyme, fatty acid synthase (Blennemann et al., Mol. Cell. Endocrinol. 110(1-2):1-8 (1995)) and CYP4A. HMG CoA reductase and LDL receptor genes have been identified as affecting cholesterol synthesis and as being responsive to T3. CPT-1 is a T3-responsive gene involved in fatty acid oxidation. Genes affecting energy expenditure, including mitochondrial genes such as mitochondrial sn-glycerol 3-phosphate dehydrogenase (mGPDH), and/or enzymes associated with proton leakage such as the adenine nucleotide transporter (ANT), Na⁺/K⁺-ATPase, Ca²⁺-ATPase and ATP synthase are also T3-responsive genes. T3-responsive genes affecting glycogenolysis and gluconeogenesis include glucose 6-phosphatase and PEPCK.

Thyroid hormone-responsive genes in the heart are not as well described as the liver but could be determined using similar techniques as described by Feng et al. Many of the genes described to be affected in the heart are the same as described above for the liver. Common genes evaluated include mitochondrial sn-glycerol 3-phosphate dehydrogenase (mGPDH), and myosin heavy and light chains (Danzi et al., Thyroid 12(6):467-72 (2002)).

Compounds used in the methods bind to thyroid receptors and produce a change in some hepatic gene expression. Evidence for agonist activity may be obtained using standard assays described in the literature.

Metabolites of the Compounds of the Invention

Also falling within the scope of the present invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammalian tissue or a mammal for a period of time sufficient to yield a metabolic product thereof. Such products typically are identified by preparing a radio-labeled (e.g. C¹⁴ or H³) compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention even if they possess no biological activity of their own.

Pharmaceutical Compositions of the Invention

While it is possible for the compounds of the present invention to be administered neat, it may be preferable to formulate the compounds as pharmaceutical compositions. As such, in yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically or prophylactically effective amount of at least one compound of the present invention, together with one or more pharmaceutically acceptable excipients. Optionally, the pharmaceutical compositions of the invention may comprise a combination of compounds of the present invention, or may include a second active ingredient useful in a method disclosed herein.

Formulations of the present invention, e.g., for parenteral or oral administration, are most typically solids, liquid solutions, emulsions or suspensions, while inhaleable formulations for pulmonary administration are generally liquids or powders, with powder formulations being generally preferred. A preferred pharmaceutical composition of the invention may also be formulated as a lyophilized solid that is reconstituted with a physiologically compatible solvent prior to administration. Alternative pharmaceutical compositions of the invention may be formulated as syrups, creams, ointments, tablets, and the like.

The pharmaceutical compositions of the invention can be administered to the subject via any drug delivery route known in the art. Specific exemplary administration routes include oral, ocular, rectal, buccal, topical, nasal, ophthalmic, subcutaneous, intramuscular, intraveneous (bolus and infusion), intracerebral, transdermal, and pulmonary.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

The therapeutically effective amount, as used herein, refers to an amount of a pharmaceutical composition of the invention to treat, ameliorate, or modulate an identified disease or condition, or to exhibit a detectable therapeutic or inhibitory effect. The effect can be detected by, for example, assays of the present invention. The effect can also be the prevention of a disease or condition where the disease or condition is predicted for an individual or a high percentage of a population.

The precise effective amount for a subject will depend upon the subject's body weight, size, and health; the nature and extent of the condition; the therapeutic or combination of therapeutics selected for administration, the protein half-life, the mRNA half-life and the protein localization. Therapeutically effective amounts for a given situation can be determined by routine experimentation that is within the skill and judgment of the clinician.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

The magnitude of a prophylactic or therapeutic dose of a particular active ingredient of the invention in the acute or chronic management of a disease or condition will vary, however, with the nature and severity of the disease or condition, and the route by which the active ingredient is administered. The dose, and perhaps the dose frequency, will also vary according to the age, body weight, and response of the individual patient. Suitable dosing regimens can be readily selected by those skilled in the art with due consideration of such factors. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

In one aspect, the compounds of the invention are administered orally in a total daily dose of about 0.375 μg/kg/day to about 3.75 mg/kg/day. In another aspect the total daily dose is from about 3.75 μg/kg/day to about 0.375 mg/kg/day. In another aspect the total daily dose is from about 3.75 μg/kg/day to about 37.5 μg/kg/day. In another aspect the total daily dose is from about 3.75 μg/kg/day to about 60 μg/kg/day. In a further aspect the dose range is from 30 μg/kg/day to 3.0 mg/kg/day. In one aspect, the compounds of the invention are administered orally in a unit dose of about 0.375 μg/kg to about 3.75 mg/kg. In another aspect the unit dose is from about 3.75 μg/kg to about 0.375 mg/kg. In another aspect the unit dose is from about 3.75 μg/kg to about 37.5 μg/kg. In another aspect the unit dose is from about 3.75 μg/kg to about 60 μg/kg. In one aspect, the compounds of the invention are administered orally in a unit dose of about 0.188 μg/kg to about 1.88 mg/kg. In another aspect the unit dose is from about 1.88 μg/kg to about 0.188 mg/kg. In another aspect the unit dose is from about 1.88 μg/kg to about 18.8 μg/kg. In another aspect the unit dose is from about 1.88 μg/kg to about 30 μg/kg. In one aspect, the compounds of the invention are administered orally in a unit dose of about 0.125 μg/kg to about 1.25 mg/kg. In another aspect the unit dose is from about 1.25 μg/kg to about 0.125 mg/kg. In another aspect the unit dose is from about 1.25 μg/kg to about 12.5 μg/kg. In another aspect the unit dose is from about 1.25 μg/kg to about 20 μg/kg. In one embodiment the unit dose is administered once a day. In another embodiment the unit dose is administered twice a day. In another embodiment the unit dose is administered three times a day. In another embodiment the unit dose is administered four times a day.

Dose refers to the equivalent of the free acid. The use of controlled-release preparations to control the rate of release of the active ingredient may be preferred. The daily dose may be administered in multiple divided doses over the period of a day. Doses and dosing schedules may be adjusted to the form of the drug or form of delivery used. For example, different dosages and scheduling of doses may be used when the form of the drug is in a controlled release form or intravenous delivery is used with a liquid form.

The phrases “therapeutically effective amount”, “prophylactically effective amount” and “therapeutically or prophylactically effective amount,” as used herein encompass the above described dosage amounts and dose frequency schedules. Different therapeutically effective amounts may be applicable for different diseases and conditions, as will be readily known by those of ordinary skill in the art. Similarly, amounts sufficient to treat or prevent such diseases, but insufficient to cause, or sufficient to reduce, adverse effects associated with conventional therapies are also encompassed by the above described dosage amounts and dose frequency schedules.

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time, protein of interest half-life, RNA of interest half-life, frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Combination Therapy

It is also possible to combine any compound of the present invention with one or more other active ingredients useful in the methods described herein, including compounds in a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the activity of the compounds of the invention.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

By way of example, the compounds of the present invention can be administered in combination with other pharmaceutical agents that are used to lower serum cholesterol such as a cholesterol biosynthesis inhibitor or a cholesterol absorption inhibitor, especially a HMG-CoA reductase inhibitor, or a HMG-CoA synthase inhibitor, or a HMG-CoA reductase or synthase gene expression inhibitor, a cholesteryl ester transfer protein (CETP) inhibitor (e.g., torcetrapib), a bile acid sequesterant (e.g., cholestyramine (Questran®), colesevelam and colestipol (Colestid®)), or a bile acid reabsorption inhibitor (see, for example, U.S. Pat. No. 6,245,744, U.S. Pat. No. 6,221,897, U.S. Pat. No. 6,277,831, EP 0683 773, EP 0683 774), a cholesterol absorption inhibitor as described (e.g., ezetimibe, tiqueside, pamaqueside or see, e.g., in WO 0250027), a PPARalpha agonist, a mixed PPAR alpha/gamma agonist such as, for example, AZ 242 (Tesaglitazar, (S)-3-(4-[2-(4-methanesulfonyloxyphenyl)ethoxy]phenyl)-2-ethoxypropionic acid), BMS 298585 (N-[(4-methoxyphenoxy)carbonyl]-N-[[4-[2-(5-methyl-2-phenyl-4-oxazolyl)ethoxy]phenyl]methyl]glycine) or as described in WO 99/62872, WO 99/62871, WO 01/40171, WO 01/40169, WO96/38428, WO 01/81327, WO 01/21602, WO 03/020269, WO 00/64888 or WO 00/64876, a MTP inhibitor such as, for example, implitapide, a fibrate, an ACAT inhibitor (e.g., avasimibe), an angiotensin II receptor antagonist, a squalene synthetase inhibitor, a squalene epoxidase inhibitor, a squalene cyclase inhibitor, combined squalene epoxidase/squalene cyclase inhibitor, a lipoprotein lipase inhibitor, an ATP citrate lyase inhibitor, lipoprotein(a) antagonist, an antioxidant or niacin (e.g., slow release niacin). The compounds of the present invention may also be administered in combination with a naturally occurring compound that act to lower plasma cholesterol levels. Such naturally occurring compounds are commonly called nutraceuticals and include, for example, garlic extract and niacin.

In one aspect, the HMG-CoA reductase inhibitor is from a class of therapeutics commonly called statins. Examples of HMG-CoA reductase inhibitors that may be used include but are not limited to lovastatin (MEVACOR; see U.S. Pat. Nos. 4,231,938; 4,294,926; 4,319,039), simvastatin (ZOCOR; see U.S. Pat. Nos. 4,444,784; 4,450,171, 4,820,850; 4,916,239), pravastatin (PRAVACHOL; see U.S. Pat. Nos. 4,346,227; 4,537,859; 4,410,629; 5,030,447 and 5,180,589), lactones of pravastatin (see U.S. Pat. No. 4,448,979), fluvastatin (LESCOL; see U.S. Pat. Nos. 5,354,772; 4,911,165; 4,739,073; 4,929,437; 5,189,164; 5,118,853; 5,290,946; 5,356,896), lactones of fluvastatin, atorvastatin (LIPITOR; see U.S. Pat. Nos. 5,273,995; 4,681,893; 5,489,691; 5,342,952), lactones of atorvastatin, cerivastatin (also known as rivastatin and BAYCHOL; see U.S. Pat. No. 5,177,080, and European Application No. EP-491226A), lactones of cerivastatin, rosuvastatin (CRESTOR; see U.S. Pat. Nos. 5,260,440 and RE37314, and European Patent No. EP521471), lactones of rosuvastatin, itavastatin, nisvastatin, visastatin, atavastatin, bervastatin, compactin, dihydrocompactin, dalvastatin, fluindostatin, pitivastatin, mevastatin (see U.S. Pat. No. 3,983,140), and velostatin (also referred to as synvinolin). Other examples of HMG-CoA reductase inhibitors are described in U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86/07054. Also included are pharmaceutically acceptable forms of the above. All of the above references are incorporated herein by reference.

Non-limiting examples of suitable bile acid sequestrants include cholestyramine (a styrene-divinylbenzene copolymer containing quaternary ammonium cationic groups capable of binding bile acids, such as QUESTRAN or QUESTRAN LIGHT cholestyramine which are available from Bristol-Myers Squibb), colestipol (a copolymer of diethylenetriamine and 1-chloro-2,3-epoxypropane, such as COLESTID tablets which are available from Pharmacia), colesevelam hydrochloride (such as WelChol Tablets (poly(allylamine hydrochloride) cross-linked with epichlorohydrin and alkylated with 1-bromodecane and (6-bromohexyl)trimethylammonium bromide) which are available from Sankyo), water soluble derivatives such as 3,3-ioene, N-(cycloalkyl)alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

In the above description, a fibrate base compound is a medicament for inhibiting synthesis and secretion of triglycerides in the liver and activating lipoprotein lipase, thereby lowering the triglyceride level in the blood. Examples include bezafibrate, beclobrate, binifibrate, ciprofibrate, clinofibrate, clofibrate, clofibric acid, ethofibrate, fenofibrate, gemfibrozil, nicofibrate, pirifibrate, ronifibrate, simfibrate and theofibrate. Such an ACAT inhibitor includes, for example: a compound having the general formula (I) disclosed in WO 92/09561 [preferably FR-129169, of which the chemical name is N-(1,2-diphenylethyl)-2-(2-octyloxyphenyl)acetamide]; a compound having the general formula (I) including a pharmacologically acceptable salt/co-crystal, ester or prodrug thereof disclosed in the Japanese Patent Publication (Kohyo) Hei 8-510256 (WO 94/26702, U.S. Pat. No. 5,491,172) {preferably CI-1011, of which the chemical name is 2,6-diisopropylphenyl-N-[(2,4,6-triisopropylphenyl)acetyl]sulfamate, and in the present invention CI-1011 including a pharmacologically acceptable salt/co-crystal, ester or prodrug thereof; a compound having the general formula (I) including a pharmacologically acceptable salt/co-crystal, ester or prodrug thereof disclosed in EP 421441 (U.S. Pat. No. 5,120,738) {preferably F-1394, of which the chemical name is (1S,2S)-2-[3-(2,2-dimethylpropyl)-3-nonylureido]cyclohexan-1-yl 3-[(4R)—N-(2,2,5,5-tetramethyl-1,-3-dioxane-4-carbonyl)amino]propionate, and in the present invention F-1394 including a pharmacologically acceptable salt/co-crystal, ester or prodrug thereof}; a compound including a pharmacologically acceptable salt/co-crystal, ester or prodrug thereof disclosed in the Japanese Patent Publication (Kohyo) 2000-500771 (WO 97/19918, U.S. Pat. No. 5,990,173) [preferably F-12511, of which the chemical name is (S)-2′,3′,5′-trimethyl-4′-hydroxy-α-dodecylthio-.alpha.-phenylacetanilide, and in the present invention F-12511 including a pharmacologically acceptable salt/co-crystal, ester or prodrug thereof]; a compound having the general formula (I) including a pharmacologically acceptable salt/co-crystal, ester or prodrug thereof disclosed in the Japanese Patent Publication (Kokai) Hei 10-195037 (EP 790240, U.S. Pat. No. 5,849,732) [preferably T-2591, of which the chemical name is 1-(3-t-butyl-2-hydroxy-5-methoxyphenyl)-3-(2-cyclohexylethyl)-3-(4-dimethylaminophenyl)urea, and in the present invention T-2591 including a pharmacologically acceptable salt/co-crystal, ester or prodrug thereof]; a compound having the general formula (I) including a pharmacologically acceptable salt/co-crystal, ester or prodrug thereof disclosed in WO 96/26948 {preferably FCE-28654, of which the chemical name is 1-(2,6-diisopropylphenyl)-3-[(4R,5R)-4,5-dimethyl-2-(4-phosphonophenyl)-1,3-dioxolan-2-ylmethyl]urea, including a pharmacologically acceptable salt/co-crystal, ester or prodrug thereof}; a compound having the general formula (I) or a pharmacologically acceptable salt thereof disclosed in the specification of WO 98/54153 (EP 987254) {preferably K-10085, of which the chemical name is N-[2,4-bis(methylthio)-6-methyl-3-pyridyl]-2-[4-[2-(oxazolo[4,5-b]pyridine-2-ylthio)ethyl]piperazin-1-yl]acetamide, including a pharmacologically acceptable salt/co-crystal, ester or prodrug thereof}; a compound having the general formula (I) disclosed in WO 92/09572 (EP 559898, U.S. Pat. No. 5,475,130) [preferably HL-004, of which the chemical name is N-(2,6-diisopropylphenyl)-2-tetradecylthioacetamide]; a compound having the general formula (I) including a pharmacologically acceptable salt/co-crystal, ester or prodrug thereof disclosed in the Japanese Patent Publication (Kokai) Hei 7-82232 (EP 718281) {preferably NTE-122, of which the chemical name is trans-1,4-bis[1-cyclohexyl-3-(4-dimethylaminophenyl)ureidomethyl]cyclohexane, and in the present invention NTE-122 includes pharmacologically acceptable salts of NTE-122}; a compound including a pharmacologically acceptable salt/co-crystal, ester or prodrug thereof disclosed in the Japanese Patent Publication (Kohyo) Hei 10-510512 (WO 96/10559) {preferably FR-186054, of which the chemical name is 1-benzyl-1-[3-(pyrazol-3-yl)benzyl]-3-[2,4-bis(methylthio)-6-methylpyridin-3-yl]urea, and in the present invention FR-186054 including a pharmacologically acceptable salt/co-crystal, ester or prodrug thereof}; a compound having the general Formula I including a pharmacologically acceptable salt/co-crystal, ester or prodrug thereof disclosed in WO 96/09287 (EP 0782986, U.S. Pat. No. 5,990,150) [preferably N-(1-pentyl-4,6-dimethylindolin-7-yl)-2,2-dimethylpropaneamide, and in the present invention including a pharmacologically acceptable salt/co-crystal, ester or prodrug thereof]; and a compound having the general formula (I) including a pharmacologically acceptable salt/co-crystal, ester or prodrug thereof disclosed in WO 97/12860 (EP 0866059, U.S. Pat. No. 6,063,806) [preferably N-(1-octyl-5-carboxymethyl-4,6-dimethylindolin-7-yl)-2,2-dimethylpropaneamide, including a pharmacologically acceptable salt/co-crystal, ester or prodrug thereof]. The ACAT inhibitor preferably is a compound selected from the group consisting of FR-129169, CI-1011, F-1394, F-12511, T-2591, FCE-28654, K-10085, HL-004, NTE-122, FR-186054, N-(1-octyl-5-carboxymethyl-4,6-dimethylindolin-7-yl)-2,2-dimethylpropaneamide (hereinafter referred as compound A), and N-(1-pentyl-4,6-dimethylindolin-7-yl)-2,2-dimethylpropaneamide (hereinafter referred as compound B), including a pharmacologically acceptable salt/co-crystal, ester or prodrug thereof. The ACAT inhibitor more preferably is a compound selected from the group consisting of CI-1011, F-12511, N-(1-octyl-5-carboxymethyl-4,6-dimethylindolin-7-yl)-2,2-dimethylpropaneamide (compound A), and N-(1-pentyl-4,6-dimethylindolin-7-yl)-2,2-dimethylpropaneamide (compound B), including a pharmacologically acceptable salt/co-crystal, ester or prodrug thereof; most preferred is N-(1-octyl-5-carboxymethyl-4,6-dimethylindolin-7-yl)-2,2-dimethylpropaneamide (compound A).

An angiotensin II receptor antagonist includes, for example, a biphenyl tetrazole compound or biphenylcarboxylic acid derivative such as: a compound having the general formula (I) including a pharmacologically acceptable salt/co-crystal, ester or prodrug thereof disclosed in the Japanese Patent Publication (Kokai) Sho 63-23868 (U.S. Pat. No. 5,138,069) {preferably losartan, of which the chemical name is 2-butyl-4-chloro-1-[2′-(1H-tetrazol-5-yl)biphenyl-4-ylmethyl]-1H-imidazol-5-methanol, and in the present invention losartan including a pharmacologically acceptable salt/co-crystal, ester or prodrug thereof}; a compound having the general formula (I) including a pharmacologically acceptable salt/co-crystal, ester or prodrug thereof disclosed in the Japanese Patent Publication (Kohyo) Hei 4-506222 (WO 91/14679) {preferably irbesartan, of which the chemical name is 2-N-butyl-4-spirocyclopentane-1-[2′-(1H-tetrazol-5-yl)biphenyl-4-ylmethyl]-2-imidazoline-5-one, and in the present invention irbesartan including a pharmacologically acceptable salt/co-crystal, ester or prodrug thereof}; a compound having the general formula (I), an ester thereof, including a pharmacologically acceptable salt/co-crystal, ester or prodrug thereof disclosed in the Japanese Patent Publication (Kokai) Hei 4-235149 (EP 433983) {preferably valsartan, of which the chemical name is (S)—N-valeryl-N-[2′-(1H-tetrazol-5-yl)biphenyl-4-ylmethyl]valine, and in the present invention valsartan including a pharmacologically acceptable salt/co-crystal, ester or prodrug thereof}; a carboxylic acid derivative having the general formula (I), including a pharmacologically acceptable salt/co-crystal, ester or prodrug thereof disclosed in the Japanese Patent Publication (Kokai) Hei 4-364171 (U.S. Pat. No. 5,196,444) {preferably candesartan, of which the chemical name is 1-(cyclohexyloxycarbonyloxy)ethyl 2-ethoxy-1-[2′-(1H-tetrazol-5-yl)biphenyl-4-ylmethyl]-1H-benzimidazole-7-carboxylate, and in the present invention candesartan including a pharmacologically acceptable salt/co-crystal, ester or prodrug thereof (TCV-116 or the like), including a pharmacologically acceptable salt/co-crystal, ester or prodrug thereof}; a carboxylic acid derivative having the general formula (I), including a pharmacologically acceptable salt/co-crystal, ester or prodrug thereof disclosed in the Japanese Patent Publication (Kokai) Hei 5-78328 (U.S. Pat. No. 5,616,599) {preferably olmesartan, of which the chemical name is (5-methyl-2-oxo-1,3-dioxolen-4-yl)methyl 4-(1-hydroxy-1-methylethyl)-2-propyl-1-[2′-(1H-tetrazol-5-yl)biphenyl-4-ylmethyl]imidazole-5-carboxylate, and in the present invention olmesartan includes carboxylic acid derivatives thereof, pharmacologically acceptable esters of the carboxylic acid derivatives (CS-866 or the like), including a pharmacologically acceptable salt/co-crystal, ester or prodrug thereof}; and a compound having the general formula (I), including a pharmacologically acceptable salt/co-crystal, ester or prodrug thereof disclosed in the Japanese Patent Publication (Kokai) Hei 4-346978 (U.S. Pat. No. 5,591,762, EP 502,314) {preferably telmisartan, of which the chemical name is 4′-[[2-n-propyl-4-methyl-6-(1-methylbenzimidazol-2-yl)-benzimidazol-1-yl]-methyl]biphenyl-2-carboxylate, including a pharmacologically acceptable salt/co-crystal, ester or prodrug thereof}. The angiotensin II receptor antagonist preferably is losartan, irbesartan, valsartan, candesartan, olmesartan, or telmisartan; more preferred is losartan or olmesartan; and most preferred is olmesartan.

In addition to being useful in treating or preventing certain diseases and disorders, combination therapy with compounds of this invention maybe useful in reducing the dosage of the second drug or agent (e.g., atorvastatin).

In addition, the compounds of the present invention can be used in combination with an apolipoprotein B secretion inhibitor and/or microsomal triglyceride transfer protein (MTP) inhibitor. Some apolipoprotein B secretion inhibitors and/or MTP inhibitors are disclosed in U.S. Pat. No. 5,919,795.

Any HMG-CoA reductase inhibitor may be employed as an additional compound in the combination therapy aspect of the present invention. The term HMG-CoA reductase inhibitor refers to a compound that inhibits the biotransformation of hydroxymethylglutaryl-coenzyme A to mevalonic acid as catalyzed by the enzyme HMG-CoA reductase. Such inhibition may be determined readily by one of skill in the art according to standard assays (e.g., Methods of Enzymology, 71: 455-509 (1981); and the references cited therein). A variety of these compounds are described and referenced below. U.S. Pat. No. 4,231,938 discloses certain compounds isolated after cultivation of a microorganism belonging to the genus Aspergillus, such as lovastatin. Also U.S. Pat. No. 4,444,784 discloses synthetic derivatives of the aforementioned compounds, such as simvastatin. Additionally, U.S. Pat. No. 4,739,073 discloses certain substituted indoles, such as fluvastatin. Further, U.S. Pat. No. 4,346,227 discloses ML-236B derivatives, such as pravastatin. In addition, EP 491,226 teaches certain pyridyldihydroxyheptenoic acids, such as rivastatin. Also, U.S. Pat. No. 4,647,576 discloses certain 6-[2-(substituted-pyrrol-1-yl)-alkyl]-pyran-2-ones such as atorvastatin. Other HMG-CoA reductase inhibitors will be known to those skilled in the art. Examples of currently or previously marketed products containing HMG-CoA reductase inhibitors include cerivastatin Na, rosuvastatin Ca, fluvastatin, atorvastatin, lovastatin, pravastatin Na and simvastatin.

Any HMG-CoA synthase inhibitor may be used as an additional compound in the combination therapy aspect of this invention. The term HMG-CoA synthase inhibitor refers to a compound that inhibits the biosynthesis of hydroxymethylglutaryl-coenzyme A from acetyl-coenzyme A and acetoacetyl-coenzyme A, catalyzed by the enzyme HMG-CoA synthase. Such inhibition may be determined readily by one of skill in the art according to standard assays (e.g., Methods of Enzymology 35: 155-160 (1975); and Methods of Enzymology, 110: 19-26 (1985); and the references cited therein). A variety of these compounds are described and referenced below. U.S. Pat. No. 5,120,729 discloses certain beta-lactam derivatives. U.S. Pat. No. 5,064,856 discloses certain spiro-lactone derivatives prepared by culturing the microorganism MF5253. U.S. Pat. No. 4,847,271 discloses certain oxetane compounds such as 11-(3-hydroxymethyl-4-oxo-2-oxetayl)-3,5,7-trimethyl-2,4-undecadienoic acid derivatives. Other HMG-CoA synthase inhibitors useful in the methods, compositions and kits of the present invention will be known to those skilled in the art.

Any compound that decreases HMG-CoA reductase gene expression may be used as an additional compound in the combination therapy aspect of this invention. These agents may be HMG-CoA reductase transcription inhibitors that block the transcription of DNA or translation inhibitors that prevent translation of mRNA coding for HMG-CoA reductase into protein. Such inhibitors may either affect transcription or translation directly, or may be biotransformed into compounds that have the aforementioned attributes by one or more enzymes in the cholesterol biosynthetic cascade or may lead to the accumulation of an isoprene metabolite that has the aforementioned activities. Such regulation is readily determined by those skilled in the art according to standard assays (Methods of Enzymology, 110: 9-19 (1985)). Several such compounds are described and referenced below; however, other inhibitors of HMG-CoA reductase gene expression will be known to those skilled in the art, for example, U.S. Pat. No. 5,041,432 discloses certain 15-substituted lanosterol derivatives that are inhibitors of HMG-CoA reductase gene expression. Other oxygenated sterols that suppress the biosynthesis of HMG-CoA reductase are discussed by E. I. Mercer (Prog. Lip. Res., 32:357-416 (1993)).

Any compound having activity as a CETP inhibitor can serve as the second compound in the combination therapy aspect of the instant invention. The term CETP inhibitor refers to compounds that inhibit the cholesteryl ester transfer protein (CETP) mediated transport of various cholesteryl esters and triglycerides from HDL to LDL and VLDL. A variety of these compounds are described and referenced below; however, other CETP inhibitors will be known to those skilled in the art. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett., 6:1951-1954 (1996), respectively.

Any ACAT inhibitor can serve as an additional compound in the combination therapy aspect of this invention. The term ACAT inhibitor refers to a compound that inhibits the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research, 24:1127 (1983). A variety of these compounds are described and referenced below; however, other ACAT inhibitors will be known to those skilled in the art. U.S. Pat. No. 5,510,379 discloses certain carboxysulfonates, while WO 96/26948 and WO 96/10559 both disclose urea derivatives having ACAT inhibitory activity.

Any compound having activity as a squalene synthetase inhibitor can serve as an additional compound in the combination therapy aspect of the instant invention. The term squalene synthetase inhibitor refers to compounds that inhibit the condensation of two molecules of famesylpyrophosphate to form squalene, a reaction that is catalyzed by the enzyme squalene synthetase. Such inhibition is readily determined by those skilled in the art according to standard methodology (Methods of Enzymology 15:393-454 (1969); and Methods of Enzymology 110: 359-373 (1985); and references cited therein). A summary of squalene synthetase inhibitors has been complied in Curr. Op. Ther Patents, 861-4, (1993). EP 0 567 026 A1 discloses certain 4,1-benzoxazepine derivatives as squalene synthetase inhibitors and their use in the treatment of hypercholesterolemia and as fungicides. EP 0 645 378 A1 discloses certain seven- or eight-membered heterocycles as squalene synthetase inhibitors and their use in the treatment and prevention hypercholesterolemia and fungal infections. EP 0 645 377 A1 discloses certain benzoxazepine derivatives as squalene synthetase inhibitors useful for the treatment of hypercholesterolemia or coronary sclerosis. EP 0 611 749 A1 discloses certain substituted amic acid derivatives useful for the treatment of arteriosclerosis. EP 0 705 607 A2 discloses certain condensed seven- or eight-membered heterocyclic compounds useful as antihypertriglyceridemic agents. WO 96/09827 discloses certain combinations of cholesterol absorption inhibitors and cholesterol biosynthesis inhibitors including benzoxazepine derivatives and benzothiazepine derivatives. EP 0 701 725 A1 discloses a process for preparing certain optically-active compounds, including benzoxazepine derivatives, having plasma cholesterol and triglyceride lowering activities.

Other compounds that are currently or previously marketed for hyperlipidemia, including hypercholesterolemia, and which are intended to help prevent or treat atherosclerosis, include bile acid sequestrants, such as colestipol HCl and cholestyramine; and fibric acid derivatives, such as clofibrate, fenofibrate, and gemfibrozil. These compounds can also be used in combination with a compound of the present invention.

It is also contemplated that the compounds of the present invention be administered with a lipase inhibitor and/or a glucosidase inhibitor, which are typically used in the treatment of conditions resulting from the presence of excess triglycerides, free fatty acids, cholesterol, cholesterol esters or glucose including, inter alia, obesity, hyperlipidemia, hyperlipoproteinemia, Syndrome X, and the like.

In a combination with a compound of the present invention, any lipase inhibitor or glucosidase inhibitor may be employed. In one aspect lipase inhibitors comprise gastric or pancreatic lipase inhibitors. In a further aspect glucosidase inhibitors comprise amylase inhibitors. Examples of glucosidase inhibitors are those inhibitors selected from the group consisting of acarbose, adiposine, voglibose, miglitol, emiglitate, camiglibose, tendamistate, trestatin, pradimicin-Q and salbostatin. Examples of amylase inhibitors include tendamistat and the various cyclic peptides related thereto disclosed in U.S. Pat. No. 4,451,455, AI-3688 and the various cyclic polypeptides related thereto disclosed in U.S. Pat. No. 4,623,714, and trestatin, consisting of a mixture of trestatin A, trestatin B and trestatin C and the various trehalose-containing aminosugars related thereto disclosed in U.S. Pat. No. 4,273,765.

A lipase inhibitor is a compound that inhibits the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid phospholipid micelles, which are subsequently absorbed at the level of the brush border of the small intestine. The micelles eventually enter the peripheral circulation as chylomicrons. Accordingly, compounds, including lipase inhibitors that selectively limit or inhibit the absorption of ingested fat precursors are useful in the treatment of conditions including obesity, hyperlipidemia, hyperlipoproteinemia, Syndrome X, and the like.

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions.

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology 92: 125 (1987).

A variety of lipase inhibitors are known to one of ordinary skill in the art. However, in the practice of the methods, pharmaceutical compositions, and kits of the instant invention, generally lipase inhibitors are those inhibitors that are selected from the group consisting of lipstatin, tetrahydrolipstatin (orlistat), FL-386, WAY-121898, Bay-N-3176, valilactone, esterastin, ebelactone A, ebelactone B and RHC 80267.

The pancreatic lipase inhibitors lipstatin, 2S, 3S, SS, 7Z, 1OZ)-5-[(S)-2-formamido-4-methyl-valeryloxy]-2-hexyl-3-hydroxy-7,1 (t-hexadecanoic acid lactone, and tetrahydrolipostatin (orlistat), 2S, 3S, 55)-5-[(S)-2-formamido-4-methyl-valeryloxy]-2-hexyl-3-hydroxy-hexadecanoic acid lactone, and the variously substituted N-formylleucine derivatives and stereoisomers thereof, are disclosed in U.S. Pat. No. 4,598,089.

The pancreatic lipase inhibitor FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813.

The pancreatic lipase inhibitor WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151.

The lipase inhibitor Bay-N-3176, N-3-trifluoromethylphenyl-N′-3-chloro-4-trifluoromethylphenylurea, and the various urea derivatives related thereto, are disclosed in U.S. Pat. No. 4,405,644.

The pancreatic lipase inhibitor valilactone, and a process for the preparation thereof by the microbial cultivation of Aetinomycetes strain MG147-CF2, are disclosed in Kitahara, et al., J. Antibiotics, 40(11): 1647-50 (1987).

The lipase inhibitor esteracin, and certain processes for the preparation thereof by the microbial cultivation of Streptomyces strain ATCC 31336, are disclosed in U.S. Pat. Nos. 4,189,438 and 4,242,453.

The pancreatic lipase inhibitors ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

The lipase inhibitor RHC 80267, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562: 205-29 (1949).

The ability of RHC 80267 to inhibit the activity of myocardial lipoprotein lipase is disclosed in Carroll et al., Lipids, 27 305-7 (1992) and Chuang et al., J. Mol. Cell Cardiol., 22: 1009-16 (1990).

In another aspect of the present invention, the compounds of Formula I can be used in combination with an additional anti-obesity agent. The additional anti-obesity agent in one aspect is selected from the group consisting of a β₃-adrenergic receptor agonist, a cholecystokinin-A agonist, a monoamine reuptake inhibitor, a sympathomimetic agent, a serotoninergic agent, a dopamine agonist, a melanocyte-stimulating hormone receptor agonist or mimetic, a melanocyte-stimulating hormone receptor analog, a cannabinoid receptor antagonist, a melanin concentrating hormone antagonist, leptin, a leptin analog, a leptin receptor agonist, a galanin antagonist, a lipase inhibitor, a bombesin agonist, a neuropeptide-Y antagonist, a thyromimetic agent, dehydroepiandrosterone or an analog thereof, a glucocorticoid receptor agonist or antagonist, an orexin receptor antagonist, a urocortin binding protein antagonist, a glucagon-like peptide-1 receptor agonist, and a ciliary neurotrophic factor.

In an additional aspect the anti-obesity agents comprise those compounds selected from the group consisting of sibutramine, fenfluramine, dexfenfluramine, bromocriptine, phentermine, ephedrine, leptin, phenylpropanolamine pseudoephedrine, {4-[2-(2-[6-aminopyridin-3-yl]-2(R)-hydroxyethylamino)ethoxy]phenyl}acetic acid, {4{2-(2-[6-aminopyridin-3-yl]-2(R)-hydroxyethylamino)ethoxy]phenyl}benzoic acid, {4-[2-(2{6-aminopyridin-3-yl]-2(R)-hydroxyethylamino)ethoxy]phenyl}propionic acid, and {4-[2-(2-[6-aminopyridin-3-yl]-2(R)-hydroxyethylamino)ethoxy]phenoxy}acetic acid.

In one aspect, the present invention concerns the prevention or treatment of diabetes, including impaired glucose tolerance, insulin resistance, insulin dependent diabetes mellitus (Type I) and non-insulin dependent diabetes mellitus (NIDDM or Type II). Also included in the prevention or treatment of diabetes are the diabetic complications, such as neuropathy, nephropathy, retinopathy or cataracts.

In one aspect the type of diabetes to be treated by the compounds of the present invention is non-insulin dependent diabetes mellitus, also known as Type II diabetes or NIDDM.

Diabetes can be treated by administering to a patient having diabetes (Type I or Type II), insulin resistance, impaired glucose tolerance, or any of the diabetic complications such as neuropathy, nephropathy, retinopathy or cataracts, a therapeutically effective amount of a compound of the present invention. It is also contemplated that diabetes be treated by administering a compound of the present invention along with other agents that can be used to prevent or treat diabetes.

Representative agents that can be used to treat diabetes in combination with a compound of the present invention include insulin and insulin analogs (e.g., LysPro insulin); GLP-1 (7-37) (insulinotropin) and GLP-1 (7-36) —NH₂. Agents that enhance insulin secretion, e.g., eblorpropamide, glibenclamide, tolbutamide, tolazamide, acetohexamide, glypizide, glimepiride, repaglinide, nateglinide, meglitinide; biguanides: metformin, phenformin, buformin; A2-antagonists and imidazolines: midaglizole, isaglidole, deriglidole, idazoxan, efaroxan, fluparoxan; other insulin secretagogues linogliride, A-4166; glitazones: ciglitazone, pioglitazone, englitazone, troglitazone, darglitazone, BRL49653; fatty acid oxidation inhibitors: clomoxir, etomoxir; a-glucosidase inhibitors: acarbose, miglitol, emiglitate, voglibose, MDL25,637, camiglibose, MDL-73,945; ˜3-agonists: BRL 35135, BRL 37344, RO 16-8714, ICI D7114, CL 316,243; phosphodiesterase inhibitors: −386,398; lipid-lowering agents benfluorex; antiobesity agents: fenfluramine; vanadate and vanadium complexes (e.g., bis(cysteinamide N-octyl)oxovanadium) and peroxovanadium complexes; amylin antagonists; glucagon antagonists; gluconeogenesis inhibitors; somatostatin analogs; antilipolytic agents: nicotinic acid, acipimox, WAG 994. Also contemplated to be used in combination with a compound of the present invention are pramlintide (Symlin®), AC 2993 and nateglinide. Any agent or combination of agents can be administered as described above.

In addition, the compounds of the present invention can be used in combination with one or more aldose reductase inhibitors, DPPIV inhibitor, glycogen phosphorylase inhibitors, sorbitol dehydrogenase inhibitors, NHE-1 inhibitors and/or glucocorticoid receptor antagonists.

Any compound having activity as a fructose-1,6-bisphosphatase (FBPase) inhibitor can serve as the second compound in the combination therapy aspect of the instant invention (e.g., 2-Amino-5-isobutyl-4-{2-[5-(N,N′-bis((S)-1-ethoxycarbonyl)ethyl)phosphonamido]furanyl}thiazoles). FBPase is a key regulatory enzyme in gluconeogenesis, the metabolic pathway by which the liver synthesizes glucose from 3-carbon precursors. The term FBPase inhibitor refers to compounds that inhibit FBPase enzyme activity and thereby block the conversion of fructose-1,6-bisphosphate, the substrate of the enzyme, to fructose 6-phosphate. FBPase inhibition can be determined directly at the enzyme level by those skilled in the art according to standard methodology (e.g., Gidh-Jain M, Zhang Y, van Poelje P D et al., J Biol Chem. 1994, 269(44): 27732-8). Alternatively, FBPase inhibition can be assessed according to standard methodology by measuring the inhibition of glucose production by isolated hepatocytes or in a perfused liver, or by measuring blood glucose lowering in normal or diabetic animals (e.g., Vincent M F, Erion M D, Gruber H E, Van den Berghe, Diabetologia. 1996, 39(10):1148-55; Vincent M F, Marangos P J, Gruber H E, Van den Berghe G, Diabetes 1991 40(10):1259-66). In some cases, in vivo metabolic activation of a compound may be required to generate the FBPase inhibitor. This class of compounds may be inactive in the enzyme inhibition screen, may or may not be active in hepatocytes, but is active in vivo as evidenced by glucose lowering in the normal, fasted rat and/or in animal models of diabetes.

A variety of FBPase inhibitors are described and referenced below; however, other FBPase inhibitors will be known to those skilled in the art. Gruber et al. U.S. Pat. No. 5,658,889 described the use of inhibitors of the AMP site of FBPase to treat diabetes; WO 98/39344 and U.S. Pat. No. 6,284,748 describe purine inhibitors; WO 98/39343 and U.S. Pat. No. 6,110,903 describe benzothiazole inhibitors to treat diabetes; WO 98/39342 and U.S. Pat. No. 6,054,587 describe indole inhibitors to treat diabetes; and WO 00/14095 and U.S. Pat. No. 6,489,476 describe heteroaromatic phosphonate inhibitors to treat diabetes. Other FBPase inhibitors are described in Wright S W, Carlo A A, Carty M D et al., J Med Chem. 2002 45(18):3865-77 and WO 99/47549.

The compounds of the present invention can also be used in combination with sulfonylureas such as amaryl, alyburide, glucotrol, chlorpropamide, diabinese, tolazamide, tolinase, acetohexamide, glipizide, tolbutamide, orinase, glimepiride, DiaBeta, micronase, glibenclamide, and gliclazide.

The compounds of the present invention can also be used in combination with antihypertensive agents. Any anti-hypertensive agent can be used as the second agent in such combinations. Examples of presently marketed products containing antihypertensive agents include calcium channel blockers, such as Cardizem, Adalat, Calan, Cardene, Covera, Dilacor, DynaCirc, Procardia XL, Sular, Tiazac, Vascor, Verelan, Isoptin, Nimotop, Norvasc, and Plendil; angiotensin converting enzyme (ACE) inhibitors, such as Accupril, Altace, Captopril, Lotensin, Mavik, Monopril, Prinivil, Univasc, Vasotec and Zestril.

Examples of compounds that may be used in combination with the compounds of the present invention to prevent or treat osteoporosis include: anti-resorptive agents including progestins, polyphosphonates, bisphosphonate(s), estrogen agonists/antagonists, estrogen, estrogen/progestin combinations, Premarin, estrone, estriol or 17α- or 17β-ethynyl estradiol); progestins including algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, fluorogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol; and bone resorption inhibiting polyphosphonates including polyphosphonates such as of the type disclosed in U.S. Pat. No. 3,683,080, the disclosure of which is incorporated herein by reference. Examples of polyphosphonates include geminal diphosphonates (also referred to as bis-phosphonates), tiludronate disodium, ibandronic acid, alendronate, resindronate zoledronic acid, 6-amino-1-hydroxy-hexylidene-bisphosphonic acid and 1-hydroxy-3(methylpentylamino)-propylidene-bisphosphonic acid. Salts, co-crystals and esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1-1,1-diphosphonic acid, and hexane-6-amino-1-hydroxy-1,1-diphosphonic acid.

Estrogen agonist/antagonist include 3-(4-(1,2-diphenyl-but-1-enyl)-phenyl)acrylic acid, tamoxifen: (ethanamine, 2-(4-(1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516, the disclosure of which is incorporated herein by reference, 4-hydroxy tamoxifen, which is disclosed in U.S. Pat. No. 4,623,660, the disclosure of which is incorporated herein by reference, raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference, toremifene: (ethanamine, 2-(4-(4-chloro-1,2-diphenyl-1-butenyl)phenoxy)-N,N-dimethyl-, (Z)—, 2-hydroxy-1,2,3-propanetricarboxylate (1:1) which is disclosed in U.S. Pat. No. 4,996,225, the disclosure of which is incorporated herein by reference, centchroman: 1-(2-((4-(-methoxy-2,2,dimethyl-3-phenyl-chroman-4-yl)-phenoxy)-ethyl)-pyrrolidine, which is disclosed in U.S. Pat. No. 3,822,287, the disclosure of which is incorporated herein by reference, levormeloxifene, idoxifene: (E)-1-(2-(4-(1-(4-iodo-phenyl)-2-phenyl-but-1-enyl)phenoxy)-ethyl)-pyrrolidinone, which is disclosed in U.S. Pat. No. 4,839,155, the disclosure of which is incorporated herein by reference, 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference, 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference, (4-(2-(2-aza-bicyclo[2.2.1]hept-2-yl)-ethoxy)-phenyl)-(6-hydroxy-2-(4-hydroxy-phenyl)benzo[b]thiophen-3-yl)-methanone which is disclosed, along with methods of preparation, in PCT publication no. WO 95/10513 assigned to Pfizer Inc, TSE-424 (Wyeth-Ayerst Laboratories) and arazoxifene, cis-6-(4-fluoro-phenyl)-5-(4-(2-piperidin-1-yl-ethoxy)-phenyl)-5,6,7,8-tetrahydro-naphthalene-2-ol; (−)-cis-6-phenyl-5-(4-(2-pyrrolidin-1-yl-ethoxy)-phenyl)-5,6,7,8-tetrahydro-naphthalene-2-ol (also known as lasofoxifene); cis-6-phenyl-5-(4-(2-pyrrolidin-1-yl-ethoxy)-phenyl)-5,6,7,8-tetrahydro-naphthalene-2-ol; cis-1-(6′-pyrrolodinoethoxy-3′-pyridyl)-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,4-tetrahydroisoquinoline; cis-6-(4-hydroxyphenyl)-5-(4-(2-piperidin-1-yl-ethoxy)-phenyl)-5,6,7,8-tetrahydro-naphthalene-2-ol; 1-(4′-pyrrolidinolethoxyphenyl)-2-phenyl-6-hydroxy-1,2,3,4-tetrahydroisoquinoline, 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs. Further anti-osteoporosis agents includes a selective androgen receptor modulator (SARM). Examples of suitable SARMs include compounds such as cyproterone acetate, chlormadinone, flutamide, hydroxyflutamide, bicalutamide, nilutamide, spironolactone, 4-(trifluoromethyl)-2(1H)-pyrrolidino[3,2-g]quinoline derivatives, 1,2-dihydropyridino[5,6-g]quinoline derivatives and piperidino[3,2-g]quinolinone derivatives. Other examples include cypterone, also known as (1b,2b)-6-chloro-1,2-dihydro-17-hydroxy-3′-H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′,a′,a′-trifluoro-3-(4-fluorophenylsulfonyl)-2-hydroxy-2-methylpropiono-m-toluidide and the trade name Casodex® is disclosed in EP-100172. The enantiomers of biclutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133, WO 01/16139, WO 02/00617, WO 02/16310, U.S. Patent Application Publication No. US 2002/0099096, U.S. Patent Application Publication No. US 2003/0022868, WO 03/011302 and WO 03/011824. All of the above references are hereby incorporated by reference herein.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds in vitro and/or in vivo. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### Preparation of Compounds of the Invention

### A: 3,5-dimethyl-4-(4′-hydroxy-3′-iso-propylbenzyl)-phenoxy]-methane sulfonic acid

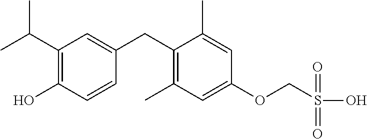

To a stirred solution of 3,5-dimethyl-4-(4′-methoxymethoxy-3′-iso-propylbenzyl)phenol (0.25 g, 0.79 mmol), (Chiellini et al., Bioorg. Med. Chem. Lett. 10:2607 (2000) in DMF (5.0 mL), was added sodium bromomethanesulfonic acid (0.32 g, 1.59 mmol), NaOH (0.31 g, 7.9 mmol). The reaction mixture was heated 150° C. in a microwave oven for 10 min and then concentrated to dryness. The crude product was dissolved in MeOH (5.0 mL) and a 30% solution of HCl in MeOH (5.0 mL) was added. After stirring at rt for 14 h, the volatiles were removed under reduced pressure. The residue was taken up in water (10 mL), extracted with ethyl acetate, dried over MgSO₄ and concentrated. The crude was purified by preparative TLC plate, eluted with CH₂Cl₂/MeOH 85/15, to afford [3,5-dimethyl-4-(4′-hydroxy-3′-iso-propylbenzyl)phenoxy]methane sulfonic acid as a white solid (130 mg, 40%): ¹H NMR (300 MHz, DMSO-d₆): δ 8.93 (s, 1H), 8.05 (bs, 1H), 6.80 (d, J=1.5 Hz, 1H), 6.65 (s, 2H), 6.54 (d, J=8.1 Hz, 1H), 6.38 (dd, J=1.5, 4.8 Hz, 1H), 4.04 (s, 2H), 3.74 (s, 2H), 3.01-3.15 (m, 1H), 2.10 (m, 6H), 1.04 (d, J=6.6 Hz, 6H); LC-MS m/z=363 [(M-1) C₁₉H₂₄O₅S]; HPLC conditions: Waters Atlantis C-18 OBD 4.6×150 mm; mobile phase=ACN/(H₂O, 0.1% TFA) flow rate=1.0 mL/min; detection=UV @ 254, 220 nm, RT=11.35 min; Anal Calcd: (MF: C₁₉H₂₄O₅S+1.2H₂O) Calcd: C, 59.11; H, 6.89; S, 8.31 Found: C, 58.90; H, 7.00; S, 8.40.

### B: 3,5-dimethyl-4-(4′-hydroxy-3′-iso-propylbenzyl)phenyl-methanesulfonic acid

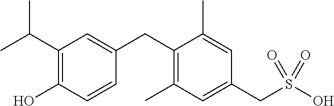

### Step a: Trifluoromethanesulfonic acid 3,5-dimethyl-4-(3′-iso-propyl-4′-methoxymethoxybenzyl)-1-phenyl ester

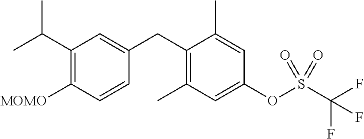

To a solution of 3,5-dimethyl-4-(3′-iso-propyl-4′-methoxymethoxybenzyl)phenol (0.6 g, 1.73 mmol) (Chiellini et al., Bioorg. Med. Chem. Lett. 10:2607 (2000)) and DMAP (0.85 g, 6.92 mmol) in CH₂Cl₂ (20 mL) at 0° C. was slowly added trifluoromethanesulfonyl anhydride (0.44 mL, 2.6 mmol). The reaction mixture was stirred at 0° C. for 2 h and quenched by water (10.0 mL). The organic layer was dried over Na₂SO₄, filtered and concentrated under reduced pressure. The crude product was purified by column chromatography on silica gel, eluting with ethyl acetate-hexanes (1:9) to afford trifluoromethanesulfonic acid 3,5-dimethyl-4-(3′-iso-propyl-4′-methoxymethoxybenzyl)-1-phenyl ester as a light yellow oil (0.83 g, 100%): ¹H NMR (300 MHz, DMSO-d₆): δ 7.09 (s, 1H), 6.87 (s, 2H), 6.80 (s, 2H), 5.15 (s, 2H), 3.81 (s, 2H), 3.36 (s, 3H), 3.20 (m, 1H), 2.20 (s, 6H), 1.14 (d, J=6.6 Hz, 6H); TLC conditions: Uniplate silica gel, 250 microns; Mobile phase=ethyl acetate-hexanes (1:9); R_{f}=0.73.

### Step b: Methyl 3,5-dimethyl-4-(4′-methoxymethoxy-3′-iso-propylbenzyl)benzoate

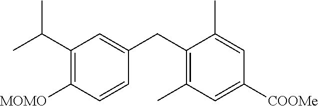

A solution of trifluoromethanesulfonic acid 3,5-dimethyl-4-(4′-methoxymethoxy-3′-iso-propylbenzyl)-phenyl ester (2.04 g, 4.57 mmol), triethylamine (1.27 mL, 9.14 mmol), 1,3-bis(diphenylphosphino)propane (0.19 mL, 0.45 mmol), MeOH (3.71 mL, 91.40 mmol), and Pd(OAc)₂ (0.102 g, 0.46 mmol) in DMF (25 mL) was heated at 90° C. under 60 psi of CO in a Parr reactor for 16 h. The reaction mixture was cooled to 0° C., diluted with ethyl acetate (25 mL) and washed with H₂O (25 mL×2). The organic solution was dried over Na₂SO₄, filtered, and concentrated under reduced pressure. The crude product was purified by column chromatography on silica gel, eluting with ethyl acetate-hexanes (1:4) to afford methyl 3,5-dimethyl-4-(4′-methoxymethoxy-3′-iso-propylbenzyl)benzoate as an oil (1.52 g, 93%): ¹H NMR (300 MHz, DMSO-d₆): δ 7.68 (s, 2H), 6.97 (m, 1H), 6.91 (m, 2H), 6.20 (m, 1H), 5.16 (s, 2H), 4.01 (s, 3H), 3.85 (s, 3H), 3.21 (m, 1H), 2.28 (s, 6H), 1.14 (d, J=6.0 Hz, 6H); TLC conditions: Uniplate silica gel, 250 microns; Mobile phase=ethyl acetate-hexanes (1:4); R_{f}=0.42.

### Step c: 3,5-dimethyl-4-(3′-iso-propyl-4′-methoxymethoxybenzyl)benzyl bromide

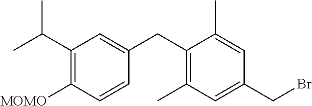

To a solution of methyl 3,5-methyl-4-(3′-isopropyl-4′-methoxymethoxybenzyl)benzoate 1.80 g, 5.0 mmol) in THF (30.0 mL) at 0° C. was slowly added DIBAL (12.6 mL, 12.6 mmol). The reaction mixture was stirred at 0° C. for 2 h and quenched with potassium sodium tartrate. The reaction mixture was diluted with hexanes and stirred at room temperature for 2 h. The organic layer was separated, dried over MgSO₄, filtered and concentrated under reduced pressure. The crude product was dissolved in ether (95.0 mL) and slowly added to a solution of carbon tetrabromide and PPh₃ in ether (20.0 mL). The reaction mixture was stirred at room temperature for 16 h and filtered through a Celite plug. The solvent was removed under reduced pressure and the crude product was purified by column chromatography on silica gel, eluting with 10% ethyl acetate in hexanes to afford 3,5-dimethyl-4-(3′-isopropyl-4′-methoxymethoxybenzyl)benzyl bromide (1.82 g, 93%) as a white solid: ¹H NMR (300 MHz, CD₃OD): δ 7.13 (s, 2H), 6.93 (m, 2H), 6.67 (d, J=7.2 Hz, 1H), 5.17 (s, 2H), 4.54 (s, 2H), 4.02 (s, 2H), 3.48 (s, 3H), 3.31 (m, 1H), 2.25 (s, 6H), 1.17 (d, J=7.0 Hz, 6H); TLC conditions: Uniplate silica gel, 250 microns; Mobile phase=ethyl acetate-hexanes (1:9); R_{f}=0.8.

### Step d: 3,5-dimethyl-4-(4′-hydroxy-3′-iso-propylbenzyl)phenyl-methanesulfonic acid

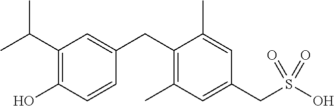

To a solution of 3,5-dimethyl-4-(3′-iso-propyl-4′-methoxymethoxybenzyl)benzyl bromide (125 mg, 0.32 mmol) in dioxane (5.0 mL) at room temperature was added a solution of Na₂SO₃ (200 mg, 1.6 mmol) in H₂O (1.0 mL). The reaction mixture was stirred at 100° C. for 10 h and cooled to room temperature. The mixture was quenched with 1 N HCl (5 mL) and extracted with ethyl acetate (2×10 mL). The organic layer was dried over MgSO₄, filtered and concentrated under reduced pressure. The crude product was dissolved in MeOH (5.0 mL) and a solution of 30% HCl in MeOH (5.0 mL) was added at rt. After stirring overnight at rt, the volatiles were removed under reduced pressure. The residue was taken up in water (10 mL), extracted with ethyl acetate, dried over MgSO₄ and concentrated. The crude mixture was purified by column chromatography on silica gel, eluting with 10CH₂Cl₂/MeOH 90/10 to afford 3,5-dimethyl-4-(4′-hydroxy-3′-iso-propylbenzyl)phenyl-methanesulfonic acid (68 mg, 40%) as light yellow solid: ¹H NMR (300 MHz, CD₃OD): δ 7.10 (s, 2H), 6.90 (d, J=1.5 Hz, 1H), 6.55 (d, J=8.1 Hz, 1H), 6.42 (dd, J=1.5, 6.3 Hz, 1H), 3.99 (s, 2H), 3.92 (s, 2H), 3.05-3.12 (m, 1H), 2.21 (s, 6H), 1.31 (d, J=6.6 Hz, 6H); TLC conditions: Uniplate silica gel, 250 microns; Mobile phase ═CH₂Cl₂/MeOH (1:3); R_{f}=0.3. LC-MS m/z=347 (M-1) [C₁₉H₂₄O₄S], HPLC conditions: Zobax SB-Aq4 6×250 nm detector wave length 254, 280 nm; mobile phase=ACN/(H₂O, 0.1% TFA) flow rate=1.0 mL/min; detection=UV @ 254, 220 nm, RT=10.92 min; Anal Calcd: (MF: C₁₉H₂₄O₄S+1.0H₂O+0.6 CH₂Cl₂) Calcd: C, 56.40; H, 6.57; S, 7.68 Found: C, 56.15; H, 6.70; S, 7.52.

### Example 3

### 3,5-dimethyl-4-(3′-iso-propyl-4′-hydroxy-benzyl) phenylsulfonic acid

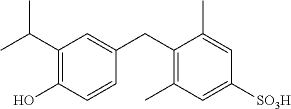

### Step a:

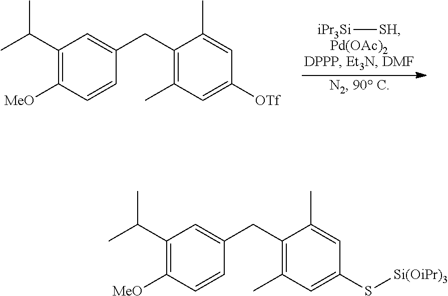

To a solution of 3,5-dimethyl-4-(3′-iso-propyl-4′-methoxy-benzyl)phenyl trifluoromethanesulfonate (2.00 g, 4.8 mmol) in DMF (24 mL) under an atmosphere of nitrogen was added triisopropylsilyl thiol (1.52 mL, 9.6 mmol), bis(diphenyphosphino)propane (200 mg, 0.48 mmol), Et₃N (1.32 mL, 9.6 mmol) and Pd(OAc)₂ (120 mg, 0.48 mmol). The reaction mixture was subsequently stirred at 90° C. for 4 h, followed by cooling to room temperature. Purification of the crude mixture by column chromatography (SiO₂, Et₂O/hexanes 0:100-5:95) afforded 3,5-dimethyl-4-(3′-iso-propyl-4′-methoxy-benzyl)phenyl triisopropylsilylsulfide as a clear oil (1.36 g, 62.3%). ¹H NMR (500 MHz, CDCl₃): δ 6.88 (s, 1H), 6.68 (m, 2H), 6.60 (s, 2H), 3.90 (s, 2H), 3.78 (s, 3H), 3.25 (sept, 1H), 2.18 (s, 6H), 1.30-1.10 (m, 27H). R_{f}=0.75 (EtOAc/hexanes 10:90).

### Step b:

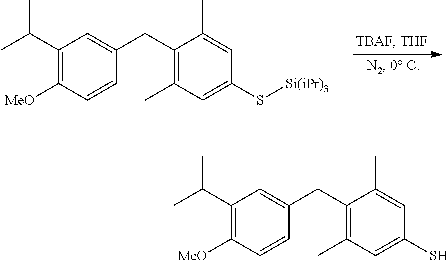

TBAF (1 M soln. in THF, 1.64 mL, 1.64 mmol) was added to a solution of 3,5-dimethyl-4-(3′-iso-propyl-4′-methoxy-benzyl)phenyl triisopropylsilylsulfide (622 mg, 1.36 mmol) in THF (13.6 mL) under an atmosphere of nitrogen at 0° C. and the reaction was stirred at 0° C. for 1 hr 25 minutes. The reaction mixture was concentrated under reduced pressure and purification by column chromatography (SiO₂, EtOAc/hexanes 10:90-40:60) afforded 3,5-dimethyl-4-(3′-iso-propyl-4′-methoxy-benzyl)thiophenol as a white solid (316 mg, 77%). ¹H NMR (500 MHz, CDCl₃): δ 6.96 (s, 1H), 6.71 (m, 2H), 6.56 (s, 2H), 3.91 (s, 2H), 3.78 (s, 3H), 3.27 (sept, 1H), 2.21 (s, 6H), 1.18 (d, J=5.5 Hz, 6H). R_{f}=0.66 (EtOAc/hexanes 10:90).

### Step c:

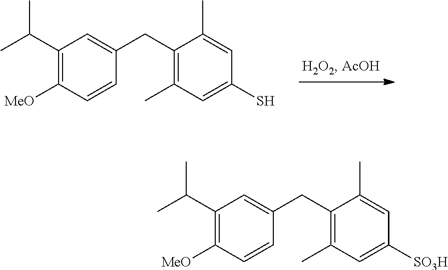

A solution of 3,5-dimethyl-4-(3′-iso-propyl-4′-methoxy-benzyl)thiophenol in H₂O₂ (30% in H₂O) and AcOH (0.8M, 5:1) is heated at reflux for 4 hours. The reaction is cooled to room temperature quenched with addition of aqueous sodium thiosulfate. The aqueous layer is extracted with EtOAc, and the combined organic layers are washed with brine, dried over Na₂SO₄ and concentrated under reduced pressure. Purification by column chromatography affords the 3,5-dimethyl-4-(3′-iso-propyl-4′-methoxy-benzyl)phenyl sulfonic acid.

### Step d:

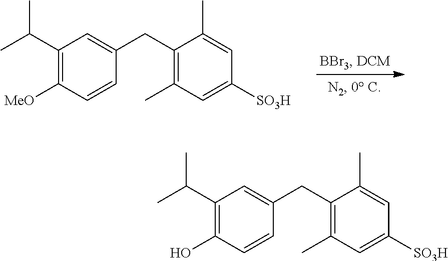

Boron tribromide is slowly added to a solution of 3,5-dimethyl-4-(3′-iso-propyl-4′-methoxy-benzyl)phenyl sulfonic acid in DCM under an atmosphere of nitrogen at 0° C. This is allowed to stir at 0° C. for 2 hours before being quenched by the addition of H₂O. The aqueous layer is extracted with DCM and the combined organic are washed with brine before concentration under reduced pressure. The crude reaction mixture is dissolved in 1M NaOH, and the aqueous layer is washed with Et₂O, acidified to pH1 by the addition of 10% HCl_{(aq.)} and extracted with EtOAc. The combined organics are then washed with brine, dried over Na₂SO₄ and concentrated under reduced pressure to afford 3,5-dimethyl-4-(3′-iso-propyl-4′-hydroxy-benzyl)phenylsulfonic acid.

### Example 2

### Activity Assays

### Receptor Binding and Oral Bioavailability

The purpose of these studies is to determine the affinity of T3 and various thyromimetics for human thyroid hormone receptors TRα and TRβ and to assess oral bioavailability.

Methods: Baculoviruses expressing TRα1, TRβ1 and RXRα are generated using cDNA and other reagents from Invitrogen (Carlsbad, Calif.). To produce TR/RXR heterodimer proteins, the sf9 insect cells are first grown to a density of 15×105 cells/mL. TRα1 or TRβ1 and RXRα baculovirus stocks are added to the cell culture with a ratio of 1 to 1 (multiplicity of infection=10). The cells are harvested three days after the infection. The cells are lysed in assay buffer (50 mM NaCl, 10% Glycerol, 20 mM tris, pH 7.6 2 mM EDTA, 5 mM β mercaptoethanol and 1.25% CHAPS) and the lysates are assayed for T3 binding as follows: ¹²⁵I-T3 is incubated with the lysates of TR and RXR recombinant baculoviruses coinfected cells (50 μl) in assay buffer for one h and then the ¹²⁵I-T3 TR/RXR complex is separated from free ¹²⁵I-T3 by a mini gel filtration (Sephadex G50) column. The bound ¹²⁵I-T3 is counted with a scintillation counter.

Binding of compounds to either the TRα1 or TRβ1 are also performed by means of scintillation proximity assays (SPA). The SPA assay, a common method used for the quantitation of receptor-ligand equilibria, makes use of special beads coated with a scintillant and a capture molecule, copper, which binds to the histidine-tagged α or β receptor. When labeled T3 is mixed with receptor and the SPA beads, radioactive counts are observed only when the complex of protein and radiolabeled ligand is captured on the surface of the bead. Displacement curves are also generated with labeled T3 and increasing concentrations of unlabeled thyromimetics of interest.

Results are shown in the table below.

| | | | | | PO 24 Hr. | |
|---|---|---|---|---|---|---|
| | | AVG | AVG | | CF TPC | PO 24 Hr. |
| Compound | | TRα Kd | TRβ Kd | | Dec (d % | TPC Dose |
| # | MOLSTRUCTURE | (nM) | (nM) | Ratio | of Cont) | (mg/kg) |
| Compound 1 | 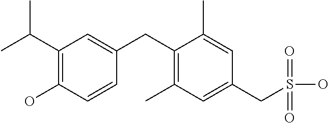 | 12.0 | 0.7 | 17.1 | −45 | 0.2 |
| Compound 2 | 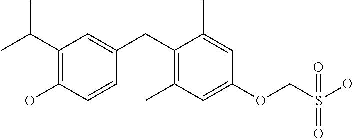 | 1.1 | 0.4 | 2.8 | −42 | 0.2 |

The oral bioavailability (OBAV) of compounds of the invention may be estimated by comparison of the dose normalized area under the curve (AUC) of the plasma concentration time profile of a compound of interest following IV and PO administration to normal rats.

Method: Groups of non-fasted male SD rats are administered 5 mg/kg of a compound of interest by IV bolus or 20 mg/kg by oral gavage. Prior to drug administration, the rats are catheterized at the tail artery to facilitate blood collection. Plasma samples are obtained at pre specified time points following dosing, extracted with 1.5 volumes of methanol, and then assayed by an LC UV method using a C18 column eluted with a gradient of 20% to 45% v/v acetonitrile in a potassium phosphate buffer pH 6.2 over 15 min with UV absorbance monitoring at 280 nm. The AUC values are determined noncompartmentally from the plasma concentration time plots by trapezoidal summation to the last measurable time point.

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

1. A compound of Formula IB: 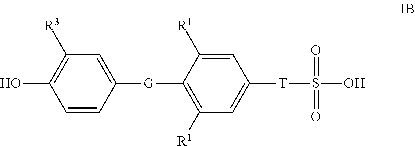
wherein:
G is selected from:

| | |
|---|---|
| —O— | —CH₂ |

T is selected from:

| | | |
|---|---|---|
| —(CR^{a}₂)ₙ— | —O(CR^{b}₂)(CR^{a}₂)ₚ— | —S(CR^{b}₂)(CR^{a}₂)ₚ— |
| —N(R^{c})(CR^{b}₂)(CR^{a}₂)ₚ— | —(CR^{b}₂)ₙN(R^{c})— | —(CR^{b}₂)ₙO— |

n is an integer from 0-2;
p is an integer from 0-1;
Each R^{a} is independently selected from:

| | | | |
|---|---|---|---|
| hydrogen | halogen | —OH | —OCF₃ |
| —OCHF₂ | —OCH₂F | —NR^{b}R^{c} | optionally |
| | | | substituted |
| | | | —C₁-C₄ alkyl |
| optionally | optionally | optionally | optionally |
| substituted | substituted | substituted | substituted |
| —O—C₁-C₄ | —S—C₁-C₄ | —C₂-C₄ | —C₂-C₄ |
| alkyl | alkyl | alkenyl | alkynyl |

with the proviso that when one R^{a} is attached to C through an O, S, or N atom, then the other R^{a} attached to the same C is a hydrogen, or attached via a carbon atom
Each R^{b} is independently selected from:

| | |
|---|---|
| hydrogen | optionally substituted C₁-C₄ alkyl |

Each R^{c} is independently selected from:

| | | | |
|---|---|---|---|
| hydrogen | —C(O)H | optionally substituted | optionally substituted |
| | | —C₁-C₄ alkyl | C(O)—C₁-C₄ alkyl |

R¹ is selected from:

| | | | |
|---|---|---|---|
| halogen | —CF₃ | cyano | optionally substituted |
| | | | —C₁-C₄ alkyl |

R³ is selected from:

| | | | |
|---|---|---|---|
| halogen | —CF₃ | —CHF₂ | —CH₂F |
| —OCF₃ | —OCHF₂ | —OCH₂F | cyano |
| —C(R^{b})═C(R^{b})— | —C(R^{b})═C(R^{b})— | C(R^{b})═C(R^{b})— | —C≡C(aryl) |
| aryl | cycloalkyl | heterocycloalkyl | |
| —C≡C(cycloalkyl) | —C≡C | —(CR^{a}₂)ₙ(CR^{b}₂)NR^{f}R^{g} | —OR^{d} |
| | (heterocycloalkyl) | | |
| —SR^{d} | —S(O)R^{e} | —S(O)₂R^{e} | —S(O)₂NR^{f}R^{g}, |
| —C(O)NR^{f}R^{g} | —C(O)OR^{h} | —C(O)R^{e} | —N(R^{b})C(O)R^{e} |
| —N(R^{b})C(O)NR^{f}R^{g} | —N(R^{b})S(O)₂R^{e} | —N(R^{b})S(O)₂NR^{f}R^{g} | —NR^{f}R^{g} |
| optionally substituted | optionally substituted | optionally substituted | optionally substituted |
| —C₁-C₁₂ alkyl | —C₂-C₁₂ alkenyl | —C₂-C₁₂ alkynyl | —(CR^{a}₂)ₘaryl |
| optionally substituted | optionally substituted | | |
| —(CR^{a}₂)ₘ cycloalkyl | —(CR^{a}₂)ₘ | | |
| | heterocycloalkyl | | |

Each R^{d} is independently selected from:

| | | | |
|---|---|---|---|
| —C(O)NR^{f}R^{g} | optionally | optionally | optionally |
| | substituted | substituted | substituted |
| | —C₁—C₁₂ | —C₂-C₁₂ | —C₂-C₁₂ |
| | alkyl | alkenyl | alkynyl |
| optionally | optionally | optionally | |
| substituted | substituted | substituted | |
| —(CR^{b}₂)ₙaryl | —(CR^{b}₂)ₙ | —(CR^{b}₂)ₙ | |
| | cycloalkyl | heterocycloalkyl | |

Each R^{e} is independently selected from:

| | | | |
|---|---|---|---|
| optionally | optionally | optionally | optionally |
| substituted | substituted | substituted | substituted |
| —C₁-C₁₂ alkyl | —C₂-C₁₂ alkenyl | —C₂-C₁₂ alkynyl | —(CR^{a}₂)ₙaryl |
| optionally | optionally | | |
| substituted | substituted | | |
| —(CR^{a}₂)ₙ | —(CR^{a}₂)ₙ | | |
| cycloalkyl | hetero- | | |
| | cycloalkyl | | |

R^{f} and R^{g} are each independently selected from:

| | | | |
|---|---|---|---|
| hydrogen | optionally | optionally | optionally |
| | substituted | substituted | substituted |
| | —C₁-C₁₂ alkyl | —C₂-C₁₂ alkenyl | —C₂-C₁₂ |
| | | | alkynyl |
| optionally | optionally | optionally | |
| substituted | substituted | substituted | |
| —(CR^{b}₂)ₙaryl | —(CR^{b}₂)ₙ | —(CR^{b}₂)ₙ | |
| | cycloalkyl | heterocycloalkyl | |

R^{f} and R^{g} may together form:
an optionally substituted heterocyclic ring of 3-8 atoms containing 0-4
unsaturations, which may contain a second heterogroup selected from the
group of O, NR^{c}, and S
wherein said optionally substituted heterocyclic ring may be substituted
with 0-4 substituents selected from the group consisting of optionally
substituted —C₁-C₄ alkyl, —OR^{b}, oxo, cyano, —CF₃, —CHF₂, —CH₂F,
optionally substituted phenyl, and —C(O)OR^{h}
Each R^{h} is selected from:

| | | | |
|---|---|---|---|
| optionally | optionally | optionally | optionally |
| substituted | substituted | substituted | substituted |
| —C₁-C₁₂ | —C₂-C₁₂ | —C₂-C₁₂ | —(CR^{b}₂)ₙaryl |
| alkyl | alkenyl | alkynyl | |
| optionally | optionally | | |
| substituted | substituted | | |
| —(CR^{b}₂)ₙcyclo- | —(CR^{b}₂)ₙhetero- | | |
| alkyl | cycloalkyl | | |

and pharmaceutically acceptable salts and prodrugs thereof and pharmaceutically acceptable salts of said prodrugs;
with the proviso that when G is —O—, —S—, —Se—, —S(═O)—, —S(═O)₂—, —CH₂—, —C(O)—, —NH—; R¹ and R² are independently chosen from the group consisting of hydrogen, halogen, —C₁-C₄ alkyl; R⁸ and R⁹ are each independently selected from hydrogen, halogen and C₁₋₄alkyl; R⁶ and R⁷ are each independently selected from hydrogen, halogen O—C₁₋₃ alkyl, hydroxy, cyano and C₁₋₄ alkyl; R³ is —C(O)NR²⁵R²⁶, —CH₂—NR²⁵R²⁶, —NR²⁵—C(O)R²⁶, —OR²⁷, R²⁸, or 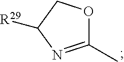
R⁴ is hydrogen, halogen, cyano or alkyl; and R⁵ is —OH; wherein R²⁵ and R²⁶ are each independently selected from the group consisting of hydrogen, aryl, heteroaryl, alkyl, cycloalkyl, aralkyl or heteroaralkyl; R²⁷ is aryl, heteroaryl, alkyl, aralkyl, or heteroaralkyl; R²⁸ is aryl, heteroaryl, or cycloalkyl; and R²⁹ is hydrogen, aryl, heteroaryl, alkyl, aralkyl, heteroaralkyl, then T may not be —(CH₂)₀₋₄— or —(CH₂)ₚ—C(O)N(R^{c})(CR^{b}₂)—; and
when G is —O—; R⁵ is —OH; R⁶, R⁷, R⁸, R⁹ are hydrogen; T is —(CH₂)ₖ—; and R⁴ is not hydrogen; then R³ may not be selected from: a substituted R²⁸—C₂-C₃ alkyl or a substituted R²⁸—C₂-C₃ alkenyl; wherein R²⁸ is aryl, heteroaryl, or cycloalkyl.

2. A compound of claim 1, wherein T is selected from:

| | | |
|---|---|---|
| —(CR^{a}₂)ₙ— | —O(CR^{b}₂)(CR^{a}₂)ₚ— | —S(CR^{b}₂)(CR^{a}₂)ₚ— |
| —(CR^{b}₂)ₙN(R^{c})— | —(CR^{b}₂)ₙO— | |

3. A compound of claim 1, wherein R¹ and R³ may each be selected from C₁ to C₄ alkyls. In other embodiments, T is preferably —(CR^{a}₂)ₙ— or —O(CR^{b}₂)(CR^{a}₂)ₚ—.

4. A compound of claim 1, selected from the group consisting of: 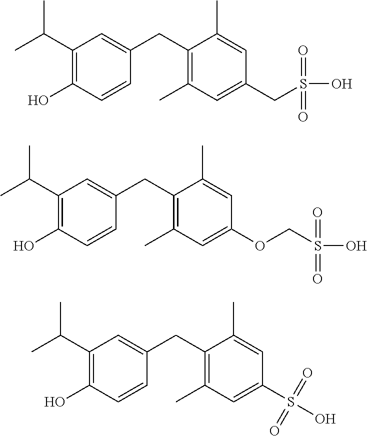
and pharmaceutically acceptable salts and prodrugs thereof and pharmaceutically acceptable salts of said prodrugs.

5. A pharmaceutical composition comprising a therapeutically effective amount of a compound of claim 1.

6. A pharmaceutical composition of claim 5 formulated as an oral dosage form.

7. A method of preventing or treating a metabolic disease in a animal in need, comprising administering to said animal in need thereof a therapeutically effective amount of a compound of claim 1, wherein said compound binds to a thyroid receptor.

8. The method of claim 7, wherein said compound binds to a thyroid receptor with a Ki of <1 μM.

9. The method of claim 8, wherein said thyroid receptor is TRα₁.

10. The method of claim 8, wherein said thyroid receptor is TRβ₁.

11. The method of claim zany of the preceding claim 7, wherein said metabolic disease is selected from the group consisting of obesity, hypercholesterolemia, hyperlipidemia, atherosclerosis, coronary heart disease, and hypertension.

12. The method of claim 11, wherein said metabolic disease is selected from the group consisting of obesity, hypercholesterolemia, and hyperlipidemia.

13. The method of claim 12, wherein said metabolic disease is hypercholesterolemia.

14. The method of claim 7, wherein said metabolic disease is fatty liver/steatosis, NAFLD, or NASH.

15. The method of claim 7, wherein said metabolic disease is selected from the group consisting of impaired glucose tolerance, diabetes, and metabolic syndrome X.

16. The method of claim 7, wherein said compound is selected from the group consisting of: 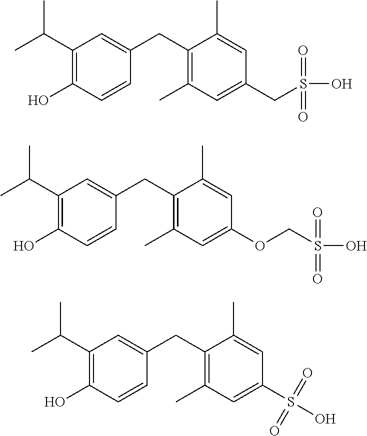
and pharmaceutically acceptable salts and prodrugs thereof and pharmaceutically acceptable salts of said prodrugs.

17. A pharmaceutical composition comprising a therapeutically effective amount of a compound of claim 4.
